SEQUENCE LISTING (4 sequences)

SEQ ID NO: 1 - protein, 28 aa, Artificial sequence
  Synthetic peptide
KLHCEKPQLGMIDRWPYHPGSFVKNREE

SEQ ID NO: 2 - protein, 25 aa, Artificial sequence
  Synthetic peptide
CEKPQLGMIDRWPYHPGSFVKNREE

SEQ ID NO: 3 - protein, 24 aa, Artificial sequence
  Synthetic peptide
CEKPQLGMIDRWYHPGSFVKNREE

SEQ ID NO: 4 - protein, 8 aa, Artificial sequence
  Synthetic peptide
SKYEKLAK

(19) United States
(12) Patent Application Publication
(10) Pub. No.: US 2022/0305015 A1
(43) Pub. Date: Sep. 29, 2022

(54) COMBINATORIAL THERAPY TARGETING PARP1 AND RTK

(71) Applicant: Board of Regents, The University of Texas System, Austin, TX (US)

(72) Inventors: Mien-Chie HUNG, Houston, TX (US); Mei-Kuang CHEN, Houston, TX (US); Yu-Yi CHU, Houston, TX (US)

(73) Assignee: Board of Regents, The University of Texas System, Austin, TX (US)

(21) Appl. No.: 17/617,923

(22) PCT Filed: Jun. 10, 2020
(86) PCT No.: PCT/US20/36900

§ 371 Date: Dec. 9, 2021

Related U.S. Application Data
(60) Provisional application No. 62/859,536, filed on Jun. 10, 2019.

(51) Int. Cl. A61K 31/5025; A61P 35/00; A61K 31/502; A61K 31/4184; A61K 31/166; A61K 31/55; A61K 31/5517; A61K 31/5377; A61K 31/473; A61K 31/454; A61K 31/519; A61K 31/496; A61K 31/53; A61K 31/4439; A61K 31/498; A61K 31/4545; A61K 31/506; A61K 45/06; C07K 16/40

(52) U.S. Cl. CPC A61K 31/5025; A61P 35/00; A61K 31/502; A61K 31/4184; A61K 31/166; A61K 31/55; A61K 31/5517; A61K 31/5377; A61K 31/473; A61K 31/454; A61K 31/519; A61K 31/496; A61K 31/53; A61K 31/4439; A61K 31/498; A61K 31/4545; A61K 31/506; A61K 45/06; C07K 16/40

(57) ABSTRACT

Provided herein are methods for identifying and treating cancers that are resistant to PARP inhibition. Methods for sensitizing cancers to a PARP inhibitor therapy are also provided. In some aspects, PARP inhibitor cancers are treated with a PARP inhibitor therapy in combination with a receptor tyrosine kinase inhibitor.

### BACKGROUND OF THE INVENTION

This application claims the benefit of U.S. Provisional Patent Application No. 62/859536, filed Jun. 10, 2019, the entirety of which is incorporated herein by reference.

Pursuant to 37 C.F.R. 1.821(c), a sequence listing is submitted herewith as an ASCII compliant text file named “UTFCP1450WO_ST25.txt”, created on Jun. 3, 2020 and having a size of ˜2 kilobytes. The content of the aforementioned file is hereby incorporated by reference in its entirety.

### FIELD OF THE INVENTION

The present invention relates generally to the fields of medicine and oncology. More particularly, it concerns methods for the identification and treatment of PARP inhibitor-resistant cancers.

### DESCRIPTION OF RELATED ART

Cancer remains a significant clinical problem. Increased levels of reactive oxygen species (ROS) in cancer cells can cause oxidative DNA damage that leads to genomic instability and tumor development (Irani et al., 1997; Trachootham et al., 2009; Radisky et al., 2005; Lindahl, 1993). ROS-induced DNA damage, such as a single-strand breaks, recruits PARP1 to the lesion sites to orchestrate the DNA repair process through poly ADP-ribosylation (PARylation) on itself and its target proteins (Luo and Kraus, 2012; Gibson and Kraus, 2012). PARP inhibitors have been widely evaluated in clinical trials since the discovery of synthetic lethality of PARP inhibition in BRCA-mutant cancer cells, which are deficient in the repair machinery of the double-strand DNA damage (Farmer et al., 2005; Bryant et al., 2005).

PARP inhibitors have been investigated in clinical trials for triple-negative breast cancer (TNBC) and may possess BRCAness properties such as BRCA mutations, methylations in the BRCA1 promoter, and dysregulation of the BRCA pathway (Hampson et al., 2010; Turashvili et al., 2011). TNBC is an aggressive subtype of breast cancer that initially responds to chemotherapy, but a majority of TNBCs eventually develop resistance. Moreover, there are no approved targeted therapies to treat TNBC, unlike other breast cancer subtypes, such as those positive for estrogen receptor (ER) and/or HER2, for which specific inhibitors are available. More than 100 clinical trials testing PARP inhibitors are underway, and the U.S. Food and Drug Administration recently approved the PARP inhibitor olaparib (Lynparza™) for the treatment of patients with BRCA-mutated ovarian cancer. While encouraging results were reported in TNBC cancer patients carrying BRCA mutations (Tutt et al., 2010), such results were not observed in another trial (Gelmon et al., 2011). These clinical observations illustrate the need to increase the response rate in TNBC and other cancer types.

While c-MET activity has been shown to enhance intrinsic resistance to PARP inhibitors in BRCA1/2 wild type triple-negative breast cancer (Du et al., 2016), its role in acquired resistance to PARP inhibitors in BRCA1/2 deficient triple-negative breast cancer remains unclear. Although co-expression of receptor tyrosine kinases might contribute to therapeutic resistance (Linklater et al., 2016), the mechanism for these interactions remains unclear. Clearly, there is a need for new and improved methods for cancer diagnosis and therapy.

### SUMMARY OF THE INVENTION

The present invention, in some aspects, overcomes limitations in the prior art by providing new methods for the treatment and diagnosis of cancers. In some aspects, it has been observed that a PARP inhibitor (PARPi) when administered with an inhibitor of a receptor tyrosine kinase (RTK inhibitor) such as for example an inhibitor of FGFR, ALK, or c-RET, can synergistically treat the cancer (e.g., a PARPi-resistant cancer, a breast cancer, a PARPi-resistant breast cancer, or a triple-negative breast cancer, etc.). As shown in the below examples, these results were observed in both in vitro and in vivo experiments. In some aspects, it has been observed that a CDK9 inhibitor can be administered to a mammalian subject in combination with a PARP(i) to synergistically treat a cancer, such as e.g., a PARPi-resistant cancer, a breast cancer, a PARPi-resistant breast cancer, or a triple-negative breast cancer. In some aspects, methods are provided that utilize testing of the phosphorylation status of PARP1 Tyr158, PARP1 Tyr176, and/or CDK9 in a cancer sample to predict resistance to a PARP1 inhibitor.

An aspect of the present invention relates to a method of treating a cancer in a mammalian subject comprising administering to the subject a therapeutically effective amount of a PARPi (e.g., a PARP1 inhibitor) and a receptor tyrosine kinase (RTK) inhibitor, wherein the RTK inhibitor is not a MET inhibitor. The RTK inhibitor may be a fibroblast growth factor receptor (FGFR) inhibitor, insulin receptor (InsR) inhibitor, Tyro3 inhibitor, anaplastic lymphoma kinase (ALK) inhibitor, Ret proto-oncogene (c-RET) inhibitor, ephrin receptor (Eph) inhibitor, RYK inhibitor, or receptor tyrosine kinase like orphan receptor (ROR) inhibitor. In some embodiments, the RTK inhibitor is a FGFR inhibitor or ALK inhibitor. In some embodiments, the patient is determined to have a cancer expressing Tyr158 and/or Try176 phosphorylated PARP1, and wherein the method comprises administering to the patient a therapeutically effective amount of a combination of a PARP1 inhibitor and an FGFR inhibitor. In some embodiments, the patient is determined to have a cancer expressing phosphorylated CDK9, and wherein the method comprises administering to the patient a therapeutically effective amount of a combination of a PARP1 inhibitor and an ALK inhibitor. In some embodiments, the cancer is a breast cancer, renal cancer, lung cancer, ovarian cancer, colon cancer, prostate cancer or pancreatic cancer. The breast cancer may be a triple-negative breast cancer. In some embodiments, the PARP1 inhibitor is olaparib, ABT-888 (Veliparib), BSI-201 (Iniparib), BMN 673, Rucaparib (AG-014699, PF-01367338), AG14361, INO-1001, A-966492, PJ34, MK-4827, or Fluzoparib. In some embodiments, the FGFR inhibitor is PD173074, AZD4547, Brivanib (BMS-540215), CHIR-258 (TKI-258), or LY2874455, Dovitanib, or JNJ42756493 (erdafitinib). In some embodiments, the ALK inhibitor is crizotinib, ceritinib, alectinib, or lorlatinib. In some embodiments, the PARP1 inhibitor is administered concurrently with or essentially simultaneously with the RTK inhibitor. In some embodiments, the patient has previously undergone at least one round of anti-cancer therapy. The subject may be a human. The method may further comprise administering a second anticancer therapy such as, e.g., a surgical therapy, chemotherapy, radiation therapy, cryotherapy, hormonal therapy, toxin therapy, immunotherapy, or cytokine therapy.

Another aspect of the present invention relates to a method of predicting resistance of a cancer in a patient to a PARP1 inhibitor comprising assaying a cancer sample to detect or determine a phosphorylation status of PARP1 Tyr158, PARP1 Tyr176, and/or CDK9 in the cancer sample; wherein increased phosphorylation of PARP1 Tyr158, PARP1 Tyr176, and/or CDK9 in the cancer sample indicates that the cancer has an increased risk of resistance to a PARP1 inhibitor. In some embodiments, PARP1 Tyr158 or Tyr176 is phosphorylated in the cancer sample. In some embodiments, CDK9 is phosphorylated in the cancer sample. The method may further comprise reporting whether the patient has a cancer that is resistant to a PARP1 inhibitor. For example, the reporting comprises preparing a written or oral report. The method may comprise reporting to the patient, a doctor, a hospital, or an insurance provider. The assaying may comprise measuring the level of phosphorylation of PARP1 Tyr158, PARP1 Tyr176, and CDK9. The assaying may comprise contacting the sample with an antibody that binds specifically to phosphorylated PARP1 Tyr158, PARP1 Tyr176, and/or CDK9. The assaying may comprise or consist of a Western blot, ELISA, immunoprecipitation, radioimmunoassay, or immunohistochemical assay. In some embodiments, the patient has a cancer that is resistant to a PARP1 inhibitor therapy, and wherein the method further comprises identifying the patient as a candidate for a combination of a PARP1 inhibitor and an RTK inhibitor. The PARP1 inhibitor may be olaparib, ABT-888 (Veliparib), BSI-201 (Iniparib), Talazoparib (BMN 673), Rucaparib (AG-014699, PF-01367338), AG14361, INO-1001, A-966492, PJ34, MK-4827, or Fluzoparib. In some embodiments, if PARP1 Tyr158 or Tyr176 is phosphorylated in the cancer sample, then the RTK inhibitor is a FGFR inhibitor. In some embodiments, the FGFR inhibitor is PD173074, AZD4547, Brivanib (BMS-540215), CHIR-258 (TKI-258), LY2874455, Dovitanib, or JNJ42756493 (erdafitinib). In some embodiments, if CDK9 is phosphorylated, then the RTK inhibitor is an ALK inhibitor. In some embodiments, the ALK inhibitor is crizotinib, ceritinib, alectinib, or lorlatinib.

Yet another aspect of the present invention relates to a method of selecting a drug therapy for a cancer patient comprising: (a) assaying cancer sample from the patient to detect or determine a phosphorylation status of PARP1 Tyr158 and/or PARP1 Tyr176 in the sample; and (b) selecting a combination of a PARP1 inhibitor and an FGFR inhibitor as the drug therapy if PARP1 Tyr158 and/or PARP1 Tyr176 is determined to be phosphorylated.

Another aspect of the present invention relates to a method of selecting a drug therapy for a cancer patient comprising: (a) assaying cancer sample from the patient to detect or determine a phosphorylation status of CDK9 in the sample; and (b) selecting a combination of a PARP1 inhibitor and an ALK inhibitor as the drug therapy if CDK9 is determined to be phosphorylated.

Yet another aspect of the present invention relates to a method of sensitizing a cancer to a PARP1 inhibitor-based anticancer therapy comprising administering an effective amount of an RTK inhibitor to a patient having the cancer, wherein the RTK inhibitor is not a MET inhibitor. The method may further comprise administering a PARP1 inhibitor-based anticancer therapy to the subject. The PARP1 inhibitor-based anticancer therapy may be administered concurrently with or essentially simultaneously with the RTK inhibitor. In some embodiments, the RTK inhibitor is a FGFR inhibitor, ALK inhibitor, TYRO3 inhibitor, InsR inhibitor, c-RET inhibitor, Eph inhibitor, RYK inhibitor, or ROR inhibitor. In some embodiments, the RTK inhibitor is a FGFR inhibitor or ALK inhibitor. The FGFR inhibitor may be PD173074, AZD4547, Brivanib (BMS-540215), CHIR-258 (TKI-258), LY2874455, Dovitanib, or JNJ42756493 (erdafitinib). In some embodiments, the ALK inhibitor is crizotinib, ceritinib, alectinib, or lorlatinib. In some embodiments, the PARP1 inhibitor is olaparib, ABT-888, BSI-201, BMN 673, Rucaparib (AG-014699, PF-01367338), AG14361, INO-1001, A-966492, PJ34, MK-4827, or Fluzoparib.

Another aspect of the present invention relates to a composition comprising a PARP1 inhibitor and an RTK inhibitor for use in treating a cancer in a patient, wherein the RTK inhibitor is not a MET inhibitor. In some embodiments, the PARP1 inhibitor is olaparib, ABT-888, BSI-201, BMN 673, Rucaparib (AG-014699, PF-01367338), AG14361, INO-1001, A-966492, PJ34, MK-4827, or Fluzoparib. In some embodiments, the RTK inhibitor is a FGFR inhibitor, ALK inhibitor, Tyro3 inhibitor, InsR inhibitor, c-RET inhibitor, Eph inhibitor, RYK inhibitor, or ROR inhibitor. In some embodiments, the RTK inhibitor is a FGFR inhibitor or ALK inhibitor. In some embodiments, the FGFR inhibitor is PD173074, AZD4547, Brivanib (BMS-540215) or CHIR-258 (TKI-258), or LY2874455, Dovitanib, or JNJ42756493 (erdafitinib). The composition may be formulated for parenteral, intravenous, intratumoral, subcutaneous, or oral administration.

Yet another aspect of the present invention relates to a composition comprising an antibody that specifically or selectively binds to either: a Tyr158-phosphorylated PARP1 protein or a Tyr176-phosphorylated PARP1 protein.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C: Activation of FGFR3 in talazoparib-resistant cells. (FIG. 1A) Colony formation of SUM149 parental cells and a pool of talazoparib-resistant SUM149 cells (SUM149-BR) in response to talazoparib (Tala). (FIG. 1B) Talazoparib half-maximal inhibitory concentration (IC₅₀) of BR cells according to the MTT assay. Fold-change (x) of IC₅₀ was compared with that of SUM149 parental cells (SUM). (FIG. 1C) Quantification of receptor tyrosine kinase antibody arrays. Cells were treated without or with talazoparib (indicated with a B) before being harvested. Signal intensities were compared with that of parental SUM149 cells.

FIGS. 2A-2C: Inhibition of FGFR3 decreases DNA damage repair. (FIG. 2A) SUM149-derived talazoparib-resistant (BR)# 17 cells were treated for 1 hour with methyl methanesulfonate (MMS), talazoparib (Tala), and PD173074 (PD) as indicated. After MMS removal, cells were cultured for the indicated time before immunofluorescence staining. Representative images of yH2AX (green) and DNA (blue) are shown. Histogram shows mean ±95% confidence interval (n >150). *p <0.05; ***p <0.001; n.s., not significant. (FIG. 2B) For comet assay analysis, BR# 09 cells were incubated for another 3 hours after MMS removal. DNA damage was normalized to that of the talazoparib-treated group. Mean ±standard deviation is shown (n >100). (FIG. 2C) Combination index (CI) of talazoparib and PD173074 in BR cells, measured by colony formation assay. Analysis of variance was used for statistical comparisons.

FIGS. 3A-3E: FGFR3 mediates PARP inhibitor resistance by phosphorylating PARP1 at Y158 residue. (FIG. 3A) Proximity ligation assay (PLA) signals of FGFR3 and PARP1 (red), phalloidin (green), and DNA (blue) were merged in representative images of the treatment indicated (MMS, methyl methanesulfonate; Tala, talazoparib; PD, PD173074; BR, SUM149-derived talazoparib-resistant cells). Mean ±95% confidence interval PLA signals in each cell nucleus are shown in the histograms (n >100). *p <0.05; ***p <0.001; n.s., not significant. (FIG. 3B) Cell survival of BR cells with PARP1 knockdown expressing hemagglutinin-tagged vector control (Neo), wild-type PARP1 (WT), or PARP1^{Y158F} (Y158F) mutant in response to talazoparib, measured by MTT assay (mean ±standard deviation; n >3). (FIG. 3C) BR# 17 cells were treated with MMS, talazoparib, and PD173074 as indicated for Western blot analysis. (FIG. 3D) Combination index (CI) of talazoparib and PD173074 in BR# 17 cells expressing PARP1^{WT} or PARP1^{Y158F} measured by MTT assay. (FIG. 3E) Cells were harvested at different time points after 40 minutes of treatment with talazoparib and MMS. Chromatin-bound PARP1 signal intensities were normalized to histone H4 and compared with that of PARP1^{WT} cells (PARP1-WT, 0 minutes). Mean ±standard deviation from 3-5 individual repeats are shown in the histograms. *p <0.05; **p <0.01; n.s., not significant. Analysis of variance was used for statistical comparisons.

FIGS. 4A-4F: Synergy of FGFR inhibitors and PARP inhibitors in xenograft models. (FIG. 4A) Mice bearing tumors consisting of SUM149-derived talazoparib-resistant (BR)# 09 cells (n=5) or BR# 17 cells (n=4) were treated with olaparib and AZD4547. Tumor volumes were measured and analysis of variance was performed (mean ±standard deviation; **p <0.01; ***p <0.001). (FIG. 4B) Survival curves for the mice shown in (A). (FIG. 4C) Tumor growth of BR# 09 (n=5) and BR# 17 (n=6) xenograft models in response to talazoparib and PD173074 (analysis of variance). (FIG. 4D) Blood chemical test of 4T1 mice treated with talazoparib and PD173074. Dot lines indicate the concentration of alanine aminotransferase (ALT), aspartate aminotransferase (AST), and blood urea nitrogen (BUN) reported in Balb/c mice. (FIG. 4E and FIG. 4F) Representative immunohistochemistry images of talazoparib-resistant and talazoparib-sensitive triple-negative breast cancer (TNBC) patient-derived xenograft models (E) and platinum-resistant and platinum-sensitive ovarian cancer (OvCa) patient-erived xenograft models (F). Scale bar, 100 μm in enlarged sections. Comparisons were made using the Mann-Whitney test (two-tailed) in GraphPad Prism 8.

FIGS. 5A-5E. Involvement of PARP1 in PARP inhibitor (PARPi)-induced cytotoxicity in SUM149-derived talazoparib-resistant cells (BR cells). (FIG. 5A) Colony formation assays of parental SUM149 cells and SUM149-BR cells in response to different concentrations of talazoparib. (FIG. 5B) Cells were treated with various concentrations of talazoparib diluted from 1 mM to 1 nM for 6 days before cell survival was measured by MTT assay (upper panel). Cells were treated with talazoparib for 10-14 days before being fixed and stained with crystal violet for colony formation assays (lower panel). The solvent (DMSO)-treated control groups were designated to have 100% survival. (FIG. 5C) Half-maximal inhibitory concentration (IC₅₀) of triple-negative breast cancer cells in response to PARPi. Cells were treated with the PARPi indicated with 10-fold dilution starting from a concentration of 1 mM and incubated for 4 days before cell survival was analyzed by MTT assay. (FIG. 5D) Olaparib, rucaparib, and veliparib IC₅₀ of parental SUM149 cells (SUM) and 31 BR cells were measured by MTT assay. Cells were treated with various concentrations of PARPi for 6 days before cell survival was analyzed by MTT assay. IC₅₀ was calculated using GraphPad Prism 8. Fold-change (x) of IC₅₀ was compared with that of SUM149 parental cells. (FIG. 5E) Knocking down PARP1 enhances talazoparib resistance in SUM149 and BR cells. Endogenous PARP1 expression was knocked down using two different short-hairpin RNAs (shRNAs) against PARP1 (shPARP1). Nontargeting shRNA was introduced as a control (Ctrl). SUM149 and BR cells expressing control shRNA or shPARP1-2 were subjected to talazoparib at various concentrations, and cell survival was measured by MTT assay.

FIGS. 6A-6C. Phosphorylated FGFR3 is higher in about half of SUM149-derived talazoparib-resistant cells (BR cells) than in SUM149 parental cells. (FIG. 6A) Antibody arrays of receptor tyrosine kinase (RTK) activation in SUM149 and BR cells. Cells indicated were treated with dimethyl sulfoxide (DMSO) or 100 nM talazoparib overnight and harvested for RTK antibody array analysis. PBS, phosphate-buffered saline. (FIG. 6B) SUM149 and 31 BR cells were treated with 100 nM talazoparib overnight before harvest for Western blot analysis. Actin was chosen to serve as a loading control. Signals of phosphorylated FGFR (p-FGFR) and FGFR3 were normalized to actin and then normalized to that of SUM149 (1-fold). Experiments were repeated three times, and the quantitation histograms indicate mean ±standard deviation. (FIG. 6C) FGFR3 expression was knocked down by short hairpin RNA (shRNA) in SUM149, BR# 09, and BR# 17 cells, and the expression of FGFR3 was examined by Western blot analysis. Survival rate of the cells indicated in response to talazoparib was analyzed by MTT assay after 6 days of treatment with talazoparib.

FIGS. 7A-7B. Activation of FGFR3 in HCC1806-BR cells. (FIG. 7A) HCC1806-derived talazoparib-resistant cells (HCC1806-BR) were more resistant to various PARP inhibitors (PARPi) than were HCC1806 parental cells. HCC1806-BR cells were developed by treating HCC1806 cells with 1₁1M talazoparib continuously for 9 months. Survival of HCC1806-BR and HCC1806 parental cells in response to talazoparib, olaparib, and rucaparib was measured by exposing cells to various concentrations of the indicated PARPi for 6 days before performing the MTT assay. (FIG. 7B) Antibody arrays of receptor tyrosine kinase activation in HCC1806 parental cells and HCC1806-BR cells. Cells were treated with either dimethyl sulfoxide (DMSO) or talazoparib overnight before being harvested for RTK antibody array analysis. PBS, phosphate-buffered saline.

FIGS. 8A-8D. Combination of talazoparib and PD173074 does not induce further DNA damages compared with talazoparib alone. (FIG. 8A) FGFR inhibitors (FGFRi) inhibited talazoparib-induced FGFR phosphorylation. SUM149-derived talazoparib-resistant cells (BR cells) and SUM149 parental cells were treated with 5μM PD173074 (PD), erdafitinib (JNJ), or AZD4547 (AZD) for 4 hours and then further exposed to 100 nM talazoparib (Tala) and 0.01% methyl methanesulfonate (MMS) in combination with the indicated FGFRi for another hour before being harvested for Western blot analysis. (FIG. 8B) BR# 17 cells were treated with 250 nM talazoparib and 0.01% MMS and FGFRi for 1 hour before being fixed and subjected to immunofluorescence staining with antibodies against FGFR3 (TexasRed, red) and yH2AX (FITC, green). DNA was counterstained with DAPI (4′6-diamidino-2-phenylindole; blue). Z-stack images were captured using confocal microscopy. (FIG. 8C) BR cells have higher DNA repair efficiency than SUM149 parental cells. SUM149, BR# 09, and BR# 17 cells were treated with 0.01% MMS for 40 minutes (+MMS) to induce DNA damage. The cells were then released from MMS by refreshing the cell culture medium to allow DNA repair for 3 hours. The untreated group (-MMS) was harvested at the same time as the groups allowed to repair for 3 hours. DNA damage was then measured by alkaline comet assay using the olive moment metric. (FIG. 8D) BR# 09 cells were treated with 0.01% MMS and 100 nM talazoparib and/or 10 μM PD173074 (either alone or in combination) for 1 hour before being harvested for alkaline comet assay analysis. Among MMS-treated groups, DNA damage was quantified using the olive moment metric and normalized to that of the talazoparib-treated group. Analysis of variance was performed, and data from individual cells are shown in each dot (n >100); means ±standard deviation are shown. ***p <0.001; n.s., not significant.

FIGS. 9A-9C. Synergism of PARP inhibitors and FGFR inhibitors in vitro. (FIG. 9A) The combination of talazoparib and PD173074 eliminates more SUM149-derived talazoparib-resistant cells (BR cells) than do single-agent treatments. Cells were treated with talazoparib (Tala) and PD173074 (PD) at the concentrations indicated for 10-12 days, and then cells were fixed for the colony formation assay. The number of colonies formed was normalized to that in the control group (not treated with talazoparib and PD173074), and mean ±standard deviation from at least three independent experiments is shown in the histogram. *p <0.05; ***p <0.001. (FIG. 9B) Combination index (CI) of the combination of talazoparib and PD173074 or olaparib and AZD4547 in multiple BR cells. Cells were treated with various concentrations of talazoparib and PD173074 combinations or olaparib and AZD4547 combinations for 6 days before cell survival was measured by MTT assay. CI was then calculated using Compusyn software and MTT data for cell survival in response to treatment. Fa, fraction affected. (FIG. 9C) CI of the talazoparib and PD173074 combination or the olaparib and AZD4547 combination in BT-549 and MDA-MB-157 cells. Cells were treated with various concentrations of talazoparib and PD173074 or olaparib and AZD4547 for 4 days before cell survival was measured by MTT assay.

FIGS. 10A-10D. FGFR3 interacts with and phosphorylates PARP1. (FIG. 10A) SUM149 parental cells and BR# 09 cells (SUM149-derived talazoparib-resistant cells) were treated with 100 nM talazoparib (PARPi) and 10 μM PD173074 (FGFRi) as indicated before being harvested for PARP1 immunoprecipitation (IP). For each sample, 500 μg total protein lysate was used for immunoprecipitation and 40 μg total protein was used for detecting target proteins in the cell lysate (input). The immunoprecipitated complex was then subjected to Western blot analysis to detect the presence of FGFR3. (FIG. 10B) FGFR phosphorylates PARP1 at Y158 and Y176 residues. His-tagged PARP1 recombinant protein was incubated with activated FGFR kinase domain before half of the samples were subjected to Western blot analysis. Phosphorylated tyrosine residues were detected by antibodies against phospho-tyrosine (clone 4G10, PY20, and PY100). The remaining samples were separated on sodium dodecyl sulfate gel, and the p120 protein band was cut from the gel and subjected to mass spectrum analysis. Phosphorylated tyrosine residues identified through mass spectrometry were aligned with the PARP1 protein sequence and marked in red. (FIG. 10C) PARP1^{Y176F} does not affect talazoparib resistance in BR cells. PARP1 knockdown (PARP1′) BR# 09 and BR# 17 cells were exogenously expressed with hemagglutinin (HA)-tagged vector control (Neo), wild-type PARP1 (WT), and PARP1^{Y158F} and PARP1^{Y176F} mutants as indicated. Exogenous PARP1 expression was examined by Western blot analysis. Cells were treated with various concentrations of talazoparib for 6 days before cell survival rate was measured by MTT assay. (FIG. 10D) Effect of PD173074 on PARP trapping. As indicated, cells were pre-treated with 10 μM PD173074 for at least 2 hours in FGFRi-treated groups before treatment with 100 nM talazoparib and 0.01% methyl methanesulfonate (MMS) for another 40 minutes. Cells were harvested after removal of MMS and talazoparib for 0, 30, or 60 minutes and subjected to cell fractionation. Chromatin-bound PARP1 was then subjected to Western blot analysis. PARP1 signal intensities were normalized to histone H4 and compared with that of cells treated with talazoparib and MMS (MMS +, PARPi +, FGFRi +, 0 minutes). Mean ±standard deviation from 3-5 individual repeats are shown in the histogram (analysis of variance). *p <0.05; **p <0.01; ***p <0.001; n.s., not significant.

FIG. 11: PARP1 Y158F mutant does not affect methyl methanesulfonate (MMS)-induced PARylation in SUM149-derived talazoparib-resistant (BR) cells. BR# 09 and BR# 17 cells expressing either wild-type PARP1 (PARP1-WT) or PARP1^{Y158F} mutant (PARP1-Y158F) were treated with 0.01% MMS for 1 hour. PARP1 expression, PARylation (PAR), and tubulin were detected by Western blot analysis. PAR signal intensities were normalized to tubulin signal intensity before being compared with wild-type PARP1-expressing cells treated with MMS (1-fold). Histograms show the mean ±standard deviation of three independent repeats. n.s., not significant.

FIGS. 12A-12F. Combination of talazoparib and PD173074 inhibits tumor growth in mouse models. (FIG. 12A) PD173074 (PD) dose titration in xenograft mouse models. SUM149-derived talazoparib-resistant cell (BR)# 09 xenograft mice (5-6 mice per group) were treated with PD173074 at 10 mg/kg or 20 mg/kg daily for 3 days before tumors were harvested for Western blot analysis. Signal intensities of phosphorylated FGFR (p-FGFR) were normalized to that of tubulin and compared with the mean intensity in a vehicle-treated group and shown in the quantitation panel (mean ±standard deviation). (FIG. 12B) Mouse body weight change (mean ±standard deviation) after treatment was normalized to that of the mouse before treatment. (FIG. 12C) Animal weights in the BR xenograft mouse models treated with 0.25 mg/kg talazoparib per day and 15 mg/kg PD173074 per day either alone or in combination. (FIG. 12D) Survival curves for the BR xenograft mouse models shown in (FIG. 12C). (FIG. 12E) 4T1 cells (500,000) were injected into the mammary fat pad of Balb/c mice. The indicated treatment was started at the time that the tumors reached an average size of 100 mm³. Because the tumor sizes varied widely within groups, all tumors were normalized to their own size on the day treatment started. Mice were then euthanized and subjected to blood chemical tests after 17 days of treatment. Mean ±standard deviation of 9 mice is shown (*p <0.05). (FIG. 12F) Animal weights in the BR xenograft mouse models treated with 40 mg/kg olaparib per day and 8 mg/kg AZD4547 per day either alone or in combination. Mean ±standard deviation of 5 mice is shown.

FIG. 13: Position of Y158 and Y176 in the PARP1 zinc finger (ZF) 2 domain. The positions of Y158 and Y176 have been emphasized in previously published crystal structures of the DNA-bound PARP1 ZF2 domain (PDB: 3ODC) (Langelieret al., 2011). Y158 and Y176 are shown here as lines, PARP1 ZF2 (blue) is shown as a cartoon backbone, zinc ion is shown as a sphere, and DNA is shown as a line.

FIG. 14: Design of PARPi resistance reference array chip. The antibodies against identified kinase/biomarkers are immobilized on an array chip. Proteins from tumor biopsies of cancer patients are extracted and applied to this array chip. If the antibody recognizes the phosphorylated kinase and/or biomarker, it will show a color change so that clinicians can quickly stratify patients to appropriate combination treatment group. This design is provided as an example, and modifications to the array chip can be made.

FIGS. 15A-15G: ALK increases protein stability and kinase activity of CDK9 through phosphorylation of Y19. (FIG. 15A) Western blot of FLAG-tagged CDK9 in cells co-expressing WT, constitutive activated, or kinase dead ALK with FLAG-tagged CDK9 after IP with indicated antibodies. (FIG. 15B) Western blot of tyrosine phosphorylation (p-Tyr) signal in in vitro kinase assay by incubating purified ALK and CDK9 protein. (FIG. 15C) Tyrosine phosphorylation (p-Tyr) signal of FLAG-tagged CDK9 was examined by western blot after IP with FLAG antibody. Cells expressed exogenous WT or Y19F CDK9 with or without constitutive activated ALK. (FIG. 15D) Expression of indicated proteins in SKOV3 stable cells expressing Y19F and WT CDK9 were examined by western blot after IP with FLAG antibody. (FIG. 15E) Western blot of FLAG-tagged CDK9 in SKOV3 stable cells expressing Y19F CDK9 and WT CDK9 treated with or without ALKi. Cells were treated with 50 μM cycloheximide (CHX) for the indicated time (left panel). Quantification of the band intensity showed in western blot (right panel). (FIG. 15F) Western blot of FLAG-tagged CDK9 in SKOV3 stable cells expressing Y19F and WT CDK9 (SEQ ID NO: 4). Cells were treated with 10 μM proteasome inhibitors (MG132 or PS-341) for the indicated time. (FIG. 15G) Ubiquitination of FLAG-tagged CDK9 in SKOV3 stable cells expressing Y19F CDK9 or WT CDK9 treated with or without ALKi.

FIGS. 16A-16C: The functional importance of p-Y19 CDK9 in ALK-mediated PARPi resistance. (FIG. 16A) Real time PCR analysis of HR repair genes expression in CDK9-knockdown SKOV3 cells rescued with WT or Y19F CDK9. (FIG. 16B) Cell viability of CDK9-knockdown SKOV3 cells rescued with WT or Y19F CDK9. Cells were plated in 24-wells and treated with the indicated concentration of talazoparib for 14 days. (FIG. 16C) Chou—Talalay analysis of CDK9-knockdown SKOV3 cells rescued with WT or Y19F CDK9. Cells were plated in 24 wells and treated with talazoparib (125 nM) or lorlatinib (1250 nM) and combined for 6 days. Synergistic inhibition of cell proliferation was defined as a combination index (CI) <1.

FIGS. 17A-17C: The combination of ALKi and PARPi effectively suppresses tumor growth in vivo. (FIGS. 17A-C) Tumor volume and Kaplan-Meier survival curves of mice bearing subcutaneous injected SKOV3, ovarian tumor (FIG. 17A) and orthotopic PARPi-resistant (acquired resistance) SUM149 tumors (# 6 and # 15) (FIGS. 17B-C). Mice were treated with oral talazoparib (0.33mg/kg) and lorlatinib (5mg/kg), either alone or in combination, five times per week.

### DESCRIPTION OF ILLUSTRATIVE EMBODIMENTS

Triple-negative breast cancer (TNBC) is a highly aggressive subtype of breast cancer that lacks expression of estrogen receptor (ER), progesterone receptor (PR), and overexpression or amplification of HER2 (Carey et al., 2010). Although some TNBC patients initially respond to chemotherapy, a majority of them eventually develop resistance (Liedtke et al., 2008). Currently, there are no effective targeted therapies against TNBC. Recently, poly (ADP-ribose) polymerase (PARP) inhibitors have emerged as promising therapeutics for patients with TNBC (Anders et al., 2010; Rouleau et al., 2010). A phase 2 study reported improved clinical benefits when the PARP inhibitor, iniparib, was combined with chemotherapy (O′Shaughnessy et al., 2011a); however, a subsequent phase 3 study indicated it did not provide significant overall survival or progression free survival benefit (O'Shaughnessy et al., 2011b) and resistance and low response rate were observed (Lord and Ashworth, 2013). Thus, it is urgent to identify potential biomarkers to stratify patients to increase the response rate of PARP inhibitor treatment. In some embodiments, a mammalian subject (e.g., a human patient) with a cancer that is or has become resistant to a PARP1 inhibitor is administered an RTK inhibitor (e.g., in combination with a PARP1 inhibitor, or before the administration of a PARP1 inhibitor). In some embodiments, administration of the RTK inhibitor can be used to re-sensitize a cancer to a PARP1 inhibitor.

PARP inhibitors (PARPi) are developed to specifically eliminate DNA damage repair (DDR) deficient tumor cells and are approved for breast and ovarian cancer treatment However, intrinsic and acquired resistances are observed in clinical and pre-clinical models. A previous study shows that c-MET kinase, a receptor tyrosine kinase (RTK) out of 20 RTK families, can phosphorylate PARP1 to cause PARPi resistance in breast cancer cells, indicating non-canonical roles of RTK in PARPi resistance. To broaden the spectrum to all RTK families in PARPi resistance, the present studies developed two sets of acquired PARPi resistant breast cancer cells from PARPi sensitive parental cell lines and identified novel RTKs that may contribute to PARPi resistance by using non-biased array screening.

The RTKs identified in the present studies include fibroblast growth factor receptor (FGFR), insulin receptor (InsR), Tyro3, anaplastic lymphoma kinase (ALK), Ret proto-oncogene (c-RET), ephrin receptor (Eph), RYK, and receptor tyrosine kinase like orphan receptor (ROR). The studies validated that FGFR receptor physically interacts with and can phosphorylate PARP1, and that FGFR inhibitors show a synergistic effect with PARPi in both in vitro and orthotopic xenograft mouse model. It was also found that the ALK receptor regulates PARPi resistance through interacting with CDK9, and the ALK inhibitor also showed synergism with PARPi in both in vitro and xenograft mouse models.

Thus, in certain embodiments, the present disclosure provides methods of inhibition of one or more of the RTK families identified herein to successfully overcome PARPi resistance in cancer treatment. In further embodiments, there is provided a method for high-throughput detection of the RTK-mediated PARPi in cancer patients by using a reference array that contains antibodies against pairs of RTK and its specific substrate (biomarker) responsive for PARPi resistance. This reference array can be used in detecting RTK-mediated PARPi resistance in patient samples and to provide a guide to determine therapeutic strategies of combining specific RTK inhibitor with PARPi to improve therapeutic response of PARPi targeted cancer.

The present studies identified combination therapies can be administered patients who do not respond to PARP inhibition. In some embodiments, the combination of PARP and RTK inhibitors are used to treat TNBC. In addition to breast cancer, these findings may also open new avenues of research on PARP inhibition in other cancer types.

In certain embodiments, the present methods concern the eight RTK families and their related-biomarkers that contribute to acquired PARPi resistance that can be used to guide kinase inhibitor and PARPi combination therapy strategies in cancer treatment. In particular, PARP1 tyrosine residue 158 and 176 can be phosphorylated by FGFR3 and can serve as biomarker for indicating FGFR-mediated PARPi resistance. Also, phosphorylated CDK9 is a biomarker for ALK-mediated PARPi resistance. Thus, also provided herein is a PARPi reference array chip comprising antibodies against the pairs of kinases and their related-biomarkers for potential clinical use to stratify patient populations. The array may include the antibody pairs of MET/Tyrosine-907 phosphorylated PARP1, FGFR3/Tyrosine-158 phosphorylated PARP1. FGFR3/Tyrosine 176-phosphorylated PARP 1 and ALK/Tyrosine phosphorylated CDK9.

The PARPi resistance reference array chip may be used to detect existing RTK-mediated PARPi resistance in patient tumor samples and thus help stratify patients to different PARPi combination treatment groups. Combining kinase inhibitors against either one or more RTKs of the RTK families with PARPi can further improve PARPi therapeutic response in breast and ovarian cancer treatments.

As shown in the below examples, cancer-associated oncogenic kinase inhibitors, which result in minimal adverse effects, were used to enhance PARPi-induced PARP trapping. To study the effects of kinases on PARP trapping and PARPi resistance, a panel of BRCAm triple-negative breast cancer cells with acquired PARPi resistance were developed, and a high prevalence of activated fibroblast growth factor receptor (FGFR) was identified among these cells. FGFR was observed to phosphorylate the PARP1 DNA binding domain to impede PARP trapping, and FGFR inhibition prolonged PARP trapping, leading to delayed DNA repair and enhanced cell death. FGFR inhibitors also synergized with PARPi to suspend cancer growth in animal models, and high FGFR phosphorylation positively correlated with PARPi resistance in patient-derived xenograft models. These findings support the idea that FGFR inhibitors can enhance the efficacy of PARPi while reducing or preventing significant adverse side effects.

### I. METHODS OF TREATING

Certain aspects of the present invention can be used to identify and/or treat a disease or disorder based on the phosphorylation state of Tyr158, and/or Tyr176 of PARP1 and/or phosphorylation of CDK9, wherein phosphorylation of these targets indicate increased risk of PARPi resistance. Other aspects of the present invention provide for sensitizing a subject with cancer to treatment with PARP inhibitors in combination with one or more RTK inhibitors.

The term “subject” or “patient” as used herein refers to any individual to which the subject methods are performed. Generally, the patient is human, although as will be appreciated by those in the art, the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

The methods described herein are useful in treating cancer. Generally, the terms “cancer” and “cancerous” refer to or describe the physiological condition in mammals that is typically characterized by unregulated cell growth. More specifically, cancers that are treated using any one or more PARP inhibitors, or variants thereof, and in connection with the methods provided herein include, but are not limited to, solid tumors, metastatic cancers, or non-metastatic cancers. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; lymphoma; blastoma; sarcoma; carcinoma, undifferentiated; giant and spindle cell carcinoma; small cell carcinoma; small cell lung cancer; non-small cell lung cancer; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous hi stiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi' s sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom' s macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Poly(ADP-ribose)polymerase 1 has an essential role in facilitating DNA repair, controlling RNA transcription, mediating cell death, and regulating immune response. PARP1 inhibitors are a group of pharmacological inhibitors of the enzyme PARP1 (see NP_001609.2, which is incorporated herein by reference). In various preclinical cancer models and human clinical trials, PARP1 inhibitors have been shown to potentiate radiation and chemotherapy by increasing apoptosis of cancer cells, limiting tumor growth, decreasing metastasis, and prolonging the survival of tumor-bearing subjects (WO 2007/084532; Donawho et al., 2007; Kummar et al., 2009). By way of example, PARP1 inhibitors include, but are not limited to, olaparib (AZD-2281), veliparib (ABT-888), iniparib (BSI-201), rucaparib (AG014699, PF-01367338), AG14361, INO-1001, A-966492, PJ34, MK-4827, CEP 9722, BNM-673, 3-aminobenzamide, fluzoparib, and those disclosed in U.S. Pat. Nos. 7,928,105; 8,124,606; 8,236,802; 8,450,323; WO 2006/110816; WO 2008/083027; and WO 2011/014681.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the prevention or treatment of disease, the appropriate dosage of a therapeutic composition, e.g., a PARP inhibitor and RTK inhibitor, will depend on the type of disease to be treated, as defined above, the severity and course of the disease, whether the agent is administered for preventive or therapeutic purposes, previous therapy, the patient's clinical history and response to the agent, and the discretion of the physician. The agent is suitably administered to the patient at one time or over a series of treatments.

A. RTK Inhibitors

Receptor tyrosine kinases (RTKs) are a family of cell surface receptors, which act as receptors for growth factors, hormones, cytokines, neurotrophic factors and other extracellular signaling molecules. RTKs mediate key signaling pathways that are involved in cell proliferation, differentiation, survival and cell migration (Lemmon and Schlessinger, 2010). The RTK family comprises several subfamilies which include, among others, epidermal growth factor receptors (EGFRs), fibroblast growth factor receptors (FGFRs), insulin and insulin-like growth factor receptors (IR and IGFR), platelet-derived growth factor receptors (PDGFRs), vascular endothelial growth factor receptors (VEGFRs), hepatocyte growth factor receptors (HGFRs), and proto-oncogene c-KIT (Li and Hristova, 2006; Hubbard and Miller, 2007). RTKs monomers are organized into an extracellular (N-terminal), a transmembrane and a cytoplasmic kinase domain. They are activated via ligand-induced dimerization that results in receptor auto-phosphorylation and tyrosine activation of RTKs' substrates including phospholipase C-γ, mitogen-activated protein kinases and phosphatidylinositol 3-kinase. In some embodiments, the RTK inhibitor is an antibody or a small molecule. In some embodiments, the RTK inhibitor is not a c-MET (MET) inhibitor or an EGFR inhibitor.

In some embodiments, the RTK inhibitor is an EGFR inhibitor. Gene mutations affecting EGFR members have been associated with several cancers, including breast cancers. Trastuzumab (Herceptin), a monoclonal antibody, can be used to target the extracellular domain of the HER2 protein in HER2-positive breast cancer patients and has been shown to increase survival at early and late stages of breast cancer. Cetuximab (Erbitux) and Panitumumab (Vectibix) are two other examples of monoclonal antibodies that can be used to target the EGFR-ligand binding. Lapatinib (Tykerb), a tyrosine kinase inhibitor, targets the ATP binding pocket of the kinase domain of EGFR and HER2 and has been used as an alternative treatment of HER2-positive breast cancer patients that developed resistance to Trastuzumab (Tripathy et al., 2004; Montemurro et al., 2006). Other EGFR inhibitors that may be used include small molecule EGFR inhibitors include such as osimertinib, gefitinib, erlotinib and brigatinib.

In some embodiments, the RTK inhibitor targets VEGF receptors (VEGFR). VEGFR have been associated with angiogenesis. A number of VEGFR inhibitors have been developed with the aim of reducing angiogenesis and lymphangiogenesis associated with cancer progression (Koch and Claesson-Welsh, 2012). For example, the VEGFR inhibitor may be a small molecule inhibitor of tyrosine protein kinase, such as Sorafenib (Nexavar®). Or Sunitinib (Sutent®, SU11248) (Motzer et al., 2009; Demetri et al., 2006). In some embodiments, the RTK inhibitor is a monoclonal antibody such as, e.g., Bevacizumab or Avastin.

The RTK inhibitor may be a PDGFR inhibitor. PDGF and PDGFRs have important functions in the regulation of cell growth and survival, and mutations in these genes have been observed in various cancers. In some embodiments the PDGFR inhibitor is a small molecule, such as for example imatinib, sunitinib, sorafenib, pazopanib or nilotinib.

In some embodiments, the RTK inhibitor is a FGFR inhibitor. Mutations in or amplifications of FGFR1 and 2 have been observed in various cancers. In some embodiments, the FGFR inhibitor is a small molecule, e.g., as described in (Huynh et al., 2008; Sarker et al., 2008; Trudel et al., 2008; Knights and Cook, 2010; Hilberg et a/.,2008; Hahn et al., 2008; Fabbro and Manley, 2001; Kumar et a/.,2007; Marek et al., 2009; and McDermott et al., 2005). In some embodiments, the FGFR inhibitor is Brivanib (BMS-540215), which is dual effect inhibitor of FGFR and VEGFR. In some embodiments, the FGFR inhibitor is CHIR-258 (TKI-258), a multiple target inhibitor (VEGFR, PDGFR, FLT-3, c-Kit and FGFR).

In some embodiments, the RTK inhibitor is a Met inhibitor. MET is the receptor for the hepatocyte growth factor and is involved in cell growth, migration, invasion, metastasis and angiogenesis. Met inhibitors that may be used include K252a, SGX523, ARQ197 (ArQule), MP470, XL880, and PF2341066 (Schiering et al., 2003; Underiner et al., 2003; Comoglio et al., 2008; Previdi et al., 2012; Bagai et al., 2010).

The RTK inhibitor may be a c-Kit inhibitor. c-Kit also known as CD117 or Mast/Stem Cell Growth Factor Receptor is a cell surface receptor of SCF (Stem Cell Factor). In some embodiments, the C-kit inhibitor is Imatinib.

Select RTK inhibitors that may be used in various embodiments are listed below in Table 1. In some embodiments, a combination of RTK inhibitors may be used. In some embodiments, a combination of an RTK inhibitor may be administered to a patient with cancer in combination with: an inhibitor of MAP kinase (MEK or Raf inhibitors) or a PI3K/AKT inhibitor.

TABLE 1
Example RTK Inhibitors

| Target | Compound |
|---|---|
| HER2 | Trastuzumab (Herceptin ®) |
| EGFR | Cetuximab (Erbitux ®) |
| | Panitumumab (Vectibix ®) |
| | Gefitinib (Iressa ®) |
| | Erlotinib (Tarceva ®) |
| EGFR and HER2 | Lapatinib (Tykerb ®) |
| | Afatinib |
| VEGFR | Sorafenib (Nexavar ®) |
| | Sunitinib (Sutent ®) |
| | Bevacizumab (Avastin ®) |
| PDGFR | Imatinib (Gleevec ®) |
| PDGFR and VEGFR | Sunitinib |
| | Soratinib |
| | Pazopanib |
| | Nilotinib |
| FGFR and VEGFR | Brivanib (BMS-540215) |
| VEGFR, PDGFR, FLT-3, | CHIR-258 (TKI-258) |
| c-KIT and FGFR | |
| MET | SGX523 |
| C-KIT | Imatinib (Gleevec ®) |

The RAS/MAP kinase pathway is involved with a variety of pathological cellular processes such as growth, proliferation, differentiation, migration and apoptosis. In some embodiments, an inhibitor of the Ras/Map kinase pathway is used. A variety of small inhibitor molecules may be used to target Mek and Raf cancers (e.g., Davies et al., 2002). In some embodiments, the RTK inhibitor is a RAS/MAP kinase pathway inhibitor such as, e.g., Sorafenib, PLX4720, PLX4032, GSK2118436, LErafAON (NeoPharm), ISIS 5132, CI-1040, PD-0325901, AZD6244, RDEA119/BAY 86-9766, GDC-0973/XL581, AZD8330/ARRY-424704, SP600125, or D-JNKI-1. Sorafenib, PLX4720, PLX4032 and GSK2118436 can be used to target B-Raf^{V600E} in malignant melanoma and other advanced malignancies. Other chemical inhibitors such as LErafAON (NeoPharm) and ISIS 5132 may target C-Raf cancers. MEK inhibitors such as CI-1040, PD-0325901, AZD6244, RDEA119/BAY 86-9766, GDC-0973/XL581 and AZD8330/ARRY-424704 can target MEK in cancers (Davies et al., 2002). Inhibitors of the JNK proteins are being investigated for potential clinical use. Inhibitors that may be used include the ATP-competitive JNK inhibitor SP600125 and JNK peptide inhibitor (D-JNKI-1) (Davies and Tournier, 2012). In some embodiments, the Ras/Map inhibitor is a 38 pathway inhibitor.

Several small molecule inhibitors of the PI3K/AKT pathway have been developed and may be used in some embodiments. For example, these inhibitors include small molecule inhibitors such as NVP-BEZ235, BGT226, XL765/SAR245409, SF1126, GDC-0980, PI-103, PF-04691502, PKI-587, and GSK2126458 (Wander et al., 2011).

B. Combination Treatments

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few h apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, a PARPi and RTK inhibitor as disclosed herein are administered concurrently or simultaneously. Nonetheless, it is envisioned that the PARPi and RTK inhibitor (e.g., inhibitor of FGFR, ALK, or c-RET) may be administered sequentially. Various combinations may be employed. For the example, in some embodiments, a PARPi is “A” and an RTK inhibitor is “B”, as follows. In some embodiments, PARPi is “A” and a CDK9 inhibitor is “B”, as follows.

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy. In some embodiments, a combination therapy disclosed herein (e.g., a PARPi in combination with an inhibitor of FGFR, ALK, c-RET, or CDK9) is administered to a mammalian subject (e.g., a human) further in combination with a chemotherapy, a radiotherapy, a immunotherapy, a checkpoint inhibitor, or a surgery.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Fibroblast Growth Factor Receptor 3 (FGFR3) is Highly Activated in Cells with Acquired PARPi Resistance

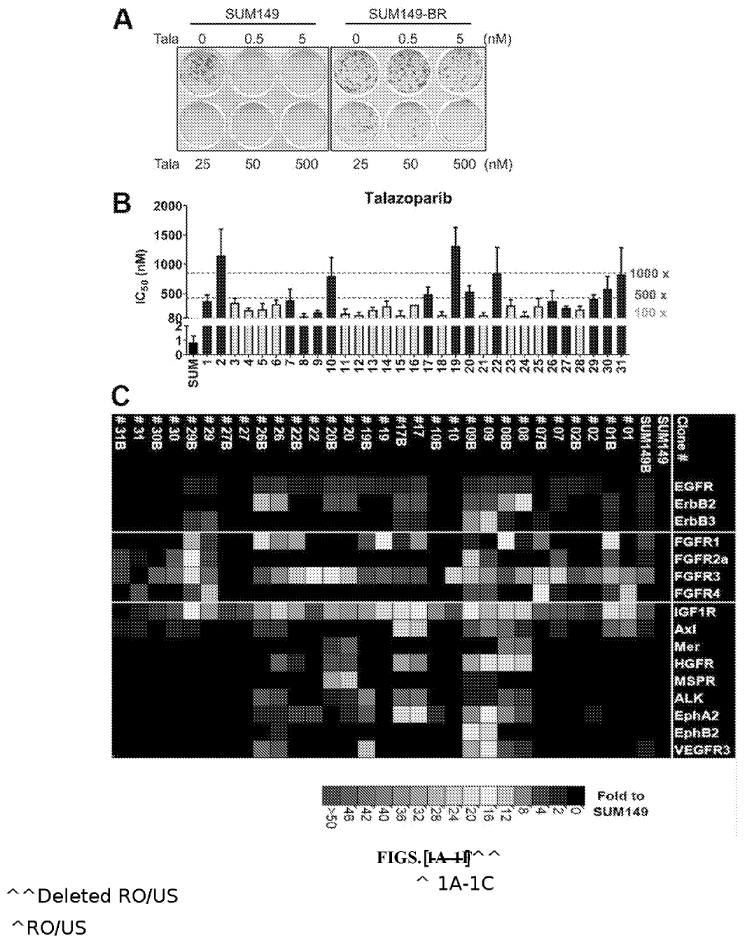

To screen for activated RTKs in PARPi-sensitive and PARPi-resistant cells with similar genetic backgrounds, a panel of 31 cell lines with acquired talazoparib resistance (BR# 01-# 31) were developed from the PARPi-sensitive BRCA //2-mutated triple-negative breast cancer (TNBC) cell line SUM149 (FIG. 5A). The half-maximal inhibitory concentration of talazoparib in the talazoparib-resistant BR cells was more than 100-fold that of the parental cells in colony formation assays and in MTT assays (FIG. 1A and FIG. 5B). As expected, the BR cells showed cross-resistance to various PARPi, including olaparib, rucaparib, and veliparib, with resistance capacity similar to that of intrinsic PARPi-resistant TNBC cells (FIG. 5C). The half-maximal inhibitory concentration of various PARPi in individual BR cells was determined, and the cells showed a range of responses to these PARPi; overall, the BR cells were more resistant to talazoparib and olaparib than to rucaparib and veliparib (FIG. 1B and FIG. 5D).

To further examine the rationale for including PARPi in TNBC treatment strategies, the inventors investigated the contribution of PARP1 to PARPi-induced cytotoxicity in SUM149, BR# 09, and BR# 17 cells. Knocking down endogenous PARP1 expression in these cells, compared with the control cells carrying non-targeting short hairpin RNA, caused at least a 10-fold increase in resistance to talazoparib (FIG. 5E), indicating that PARP1 is required for PARPi-induced cytotoxicity in these cells. Therefore, these results support the idea that PARPi resistance in these cells is not due mainly to PARPi efflux or loss of PARP1.

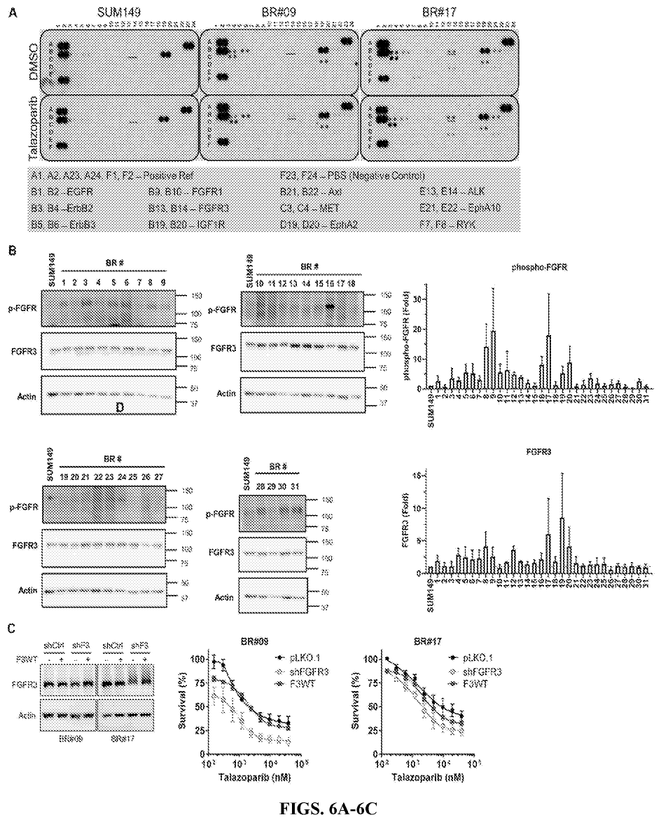

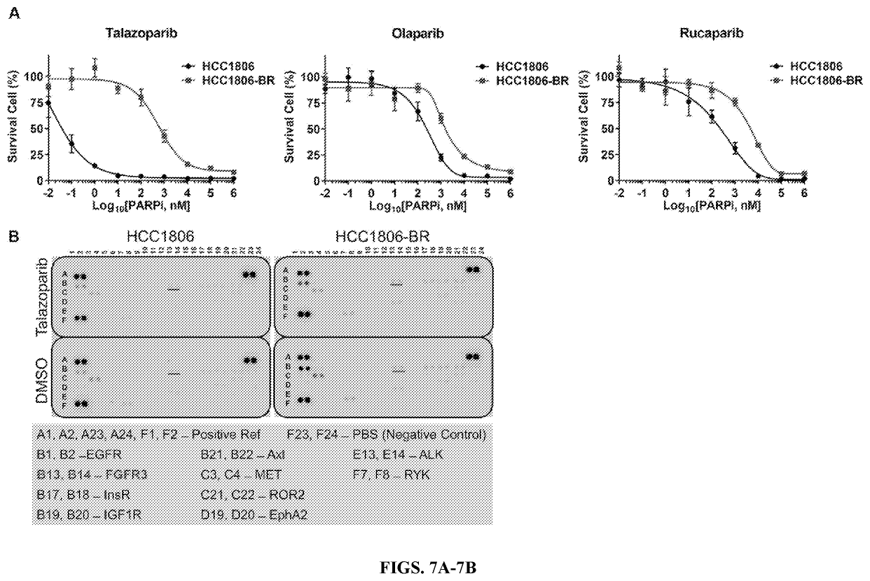

The inventors selected 15 BR cells in which to identify specific RTK activations that are harbored in these cells but not in SUM149 parental cells, using phospho-RTK antibody arrays (FIG. 6A). Quantification data from the arrays showed that phosphorylated FGFR3 had the highest prevalence of array signals that were at least 10-fold higher than in parental SUM149 cells (FIG. 1C). The array data was validated by Western blot analysis and found that phosphorylated FGFR3 and FGFR3 expression were higher in about 50% of the BR cells than in parental SUM149 cells (FIG. 6B). Furthermore, FGFR3 was also activated in HCC1806 TNBC cells with acquired talazoparib resistance (FIG. 7A-B). These results indicated that phosphorylated FGFR3 is common among TNBC cells with acquired PARPi resistance.

Two BR cells that had high FGFR3 expression (BR# 09 and BR# 17) were chosen for further study of the effects of FGFR3 on PARPi resistance. To validate the involvement of FGFR3 in PARPi resistance, endogenous FGFR3 expression was knocked down in BR# 09 and BR# 17 cells. FGFR3 knockdown strengthened talazoparib sensitivity in these cells, and FGFR3 knockdown cells rescued with wild-type FGFR3 (FGFR3^{WT}) exhibited restored resistance to talazoparib (FIG. 6C).

### Example 2

### FGFR Inhibitors (FGFRi) Impede DNA Repair Efficiency and have Synergy with PARPi

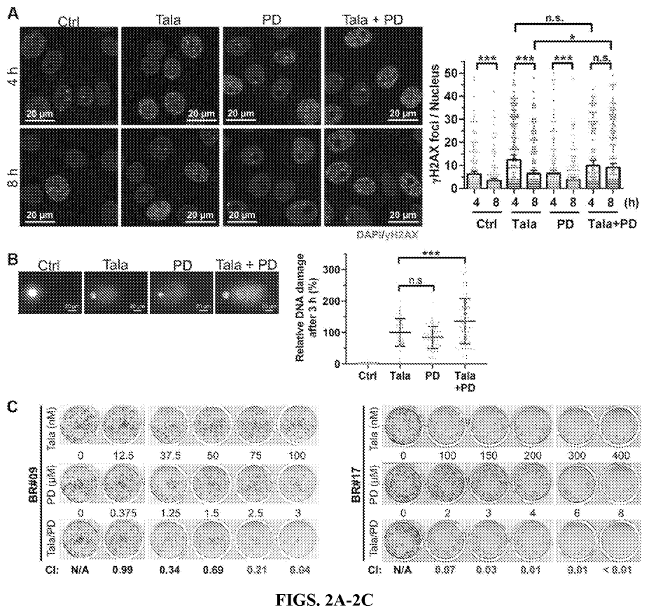

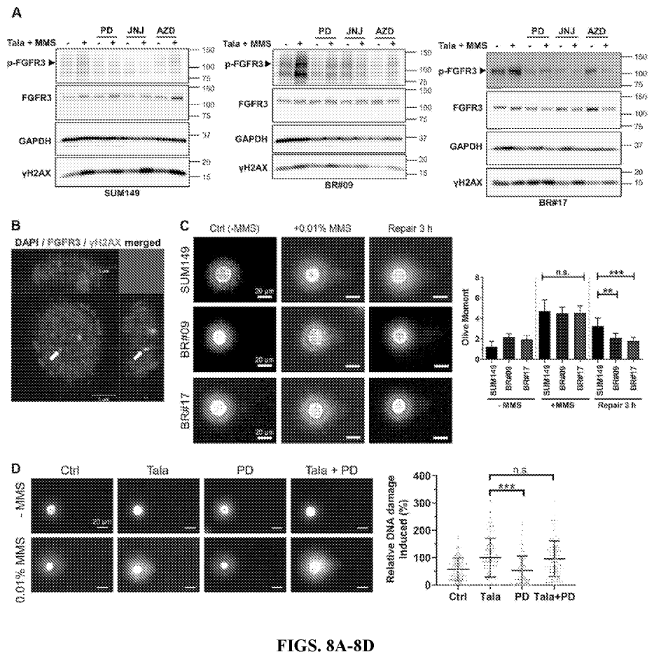

To elucidate the role of FGFR3 in PARPi resistance, the inventors first examined whether PARP1-activating DNA alkylating damage can induce FGFR3 phosphorylation. Both BR# 09 and BR# 17 cells had elevated FGFR3 phosphorylation compared with SUM149 parental cells in response to treatment with methyl methanesulfonate (MMS) and talazoparib, and the FGFR phosphorylation was inhibited by FGFRi, such as PD173074, AZD4547, and erdafitinib (FIG. 8A). Confocal microscopy imaging showed that FGFR3 co-localized with yH2AX DNA break foci in response to MMS and talazoparib (FIG. 8B), indicating that FGFR3 may affect DNA repair. yH2AX foci staining also showed that the amount of yH2AX foci formed in talazoparib-treated cells was similar to that in cells treated with the combination of talazoparib and PD173074 (FIG. 2A). Moreover, yH2AX foci significantly decreased after 8 hours of treatment compared with 4 hours of treatment (p <0.001) in untreated cells, as well as compared with cells treated with either talazoparib or PD173074 alone, but the amount of yH2AX foci remained unrepaired at 8 hours of treatment compared with 4 hours of treatment in the combination treatment group (FIG. 2A).

The same phenomenon was also observed in comet assay analyses. The combination of PD173074 and talazoparib resulted in a similar amount of DNA damage to that observed after treatment with talazoparib in BR cells (FIG. 8C-D). Although most cells in the control group eliminated DNA damage at 3 hours after MMS removal, BR cells in the talazoparib-treated group had similar unrepaired DNA damage to that observed in the PD173074-treated group, and that more DNA damage remained in the talazoparib and PD173074 combination group (FIG. 2B). The comet assay and yH2AX foci staining data both suggest that the combination treatment did not induce more DNA damage than talazoparib alone, but the combination of talazoparib and PD173074 delayed DNA repair efficiency; thus, the combination may be more cytotoxic owing to sustained burden of DNA breaks.

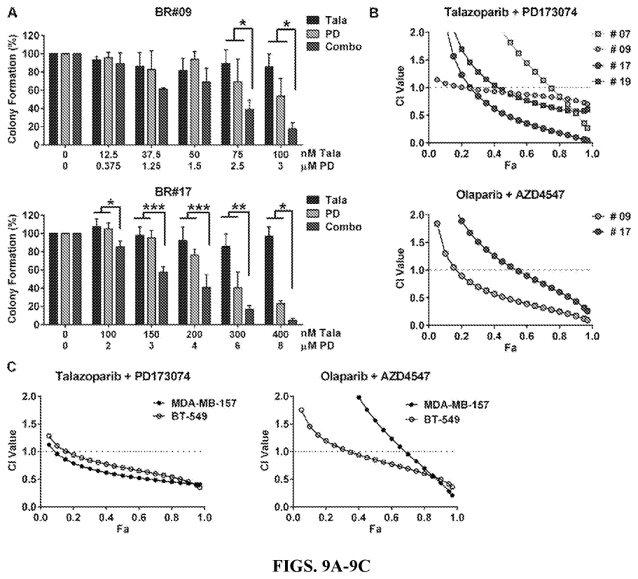

The efficacy of the combination of PARPi and FGFRi in eliminating PARPi-resistant cells was further examined. The combination index (CI) of FGFRi and PARPi was used as an indicator for synergy (CI <1) (Chou, 2010). Considering the potential for future clinical trial applications, the inventors paired inhibitors developed by the same pharmaceutical companies, e.g., talazoparib combined with PD173074 and olaparib combined with AZD4547. Using the colony formation assay, the combination of talazoparib and PD173074 had moderate synergy in BR# 09 cells and strong synergy in BR# 17 cells (FIG. 2C and FIG. 9A). The MTT assay was used to evaluate the synergy of PARPi and FGFRi in BR cells and PARPi-resistant TNBC cells. In BR cells, both the combination of talazoparib and PD173074 and the combination of olaparib and AZD4547 had moderate to strong synergy (CI between 0.1 and 0.8 when eliminating more than 80% of the cells; FIG. 9B). A CI between 0.2 and 0.8 was also reached in BT-549 and MDA-MB-157 cells, which also have endogenous FGFR3 phosphorylation (Smith et al., 2017), with combinations of FGFRi and PARPi (FIG. 9C), suggesting that the synergy of these combinations is not limited to BR cells with acquired PARPi resistance.

### Example 3

### Inhibiting FGFR-Mediated PARP1 Tyrosine 158 Phosphorylation Reverses Resistance to PARPi

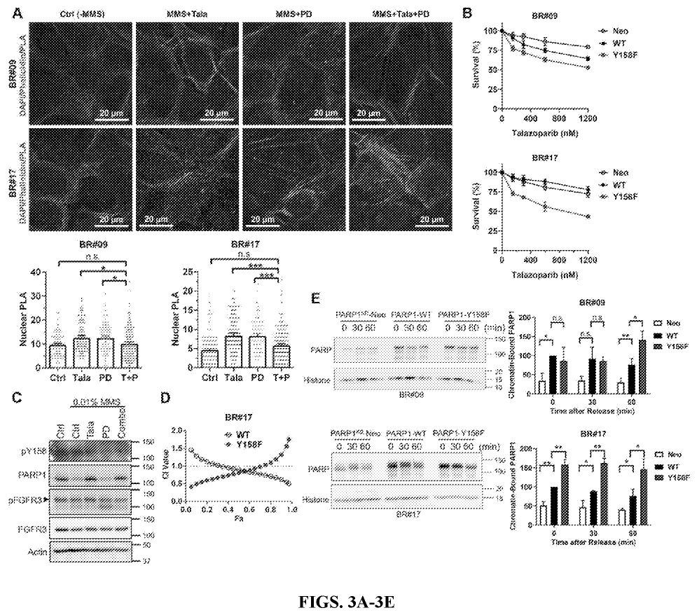

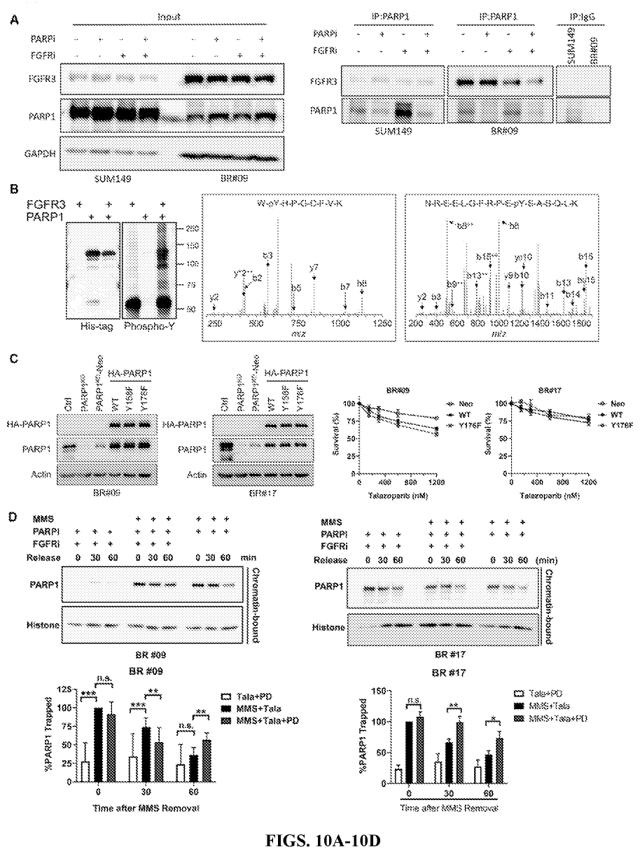

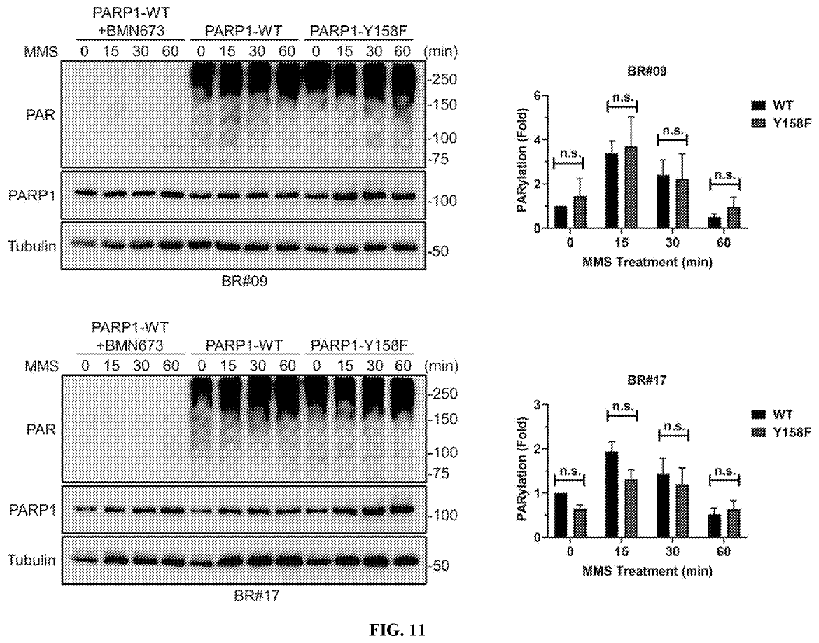

Because FGFR inhibition decreases DNA repair (FIG. 2A-B), the inventors investigated the involvement of FGFR3 in PARP1-interacting proteins. The inventors found that FGFR3 can be co-immunoprecipitated with PARP1 (FIG. 10A), and proximity ligation assay data suggested that FGFR3 and PARP1 can interact in the cell nucleus (FIG. 3A). Moreover, the proximity ligation assay signal was lower in cells treated with the combination of talazoparib and PD173074 than in cells treated with either inhibitor alone (FIG. 3A), indicating that both phosphorylated FGFR3 and activated PARP1 contribute to this interaction.

Following the proximity ligation assay results, the inventors further hypothesized that FGFR3 may phosphorylate PARP1. Through in vitro kinase assay and mass spectrum analysis, FGFR can phosphorylate PARP1 at tyrosine (Y)158 and Y176 amino acids (FIG. 10B). Y-to-phenylalanine (F)—mutated PARP1 were generated to mimic un-phosphorylated PARP1 to further examine contributions of these phosphorylation sites to PARPi resistance. MTT assay results indicated that PARP1^{Y158F} BR cells had talazoparib half-maximal inhibitory concentration values lower than those of PARP1′ cells (FIG. 3B). However, PARP1′ did not show a significant impact on cell survival in response to talazoparib (FIG. 10C). The existence of Y158-phosphorylated PARP1 (p-Y158 PARP1) in BR cells was validated using a monoclonal antibody against p-Y158 PARP1, and p-Y158 PARP1 can be diminished by PD173074 (FIG. 3C). Moreover, synergy between talazoparib and PD173074 was lower in BR cells carrying the PARP1^{Y158F} mutation (FIG. 3D). These results support the idea that FGFR3-mediated p-Y158 PARP1 inhibition plays an important role in PARPi resistance.

Because Y158 locates in DNA binding zinc finger domain (ZF) 2 of PARP1, the phosphorylation status of Y158 may affect PARP trapping. Therefore, the effect of p-Y158 PARP1 on PARP trapping using PARP1^{WT} and PARP1^{Y158F} BR cells was further examined. More PARP1 was observed to be bound to chromatin in PARP1^{Y158F}-expressing cells than in PARP1^{WT}-expressing cells (FIG. 3E), supporting the hypothesis that p-Y158-PARP1 is less vulnerable to talazoparib-mediated PARP trapping, and that FGFR3 activation enhances PARPi resistance by reducing PARP trapping. PD173074 can also prolong talazoparib-induced PARP1 trapping in BR cells (FIG. 10D). However, MMS is still capable of increasing PARylation signals in both PARP1^{WT}- and PARP1^{Y158F}-expressing BR cells, and that PARP1^{WT} and PARP1^{Y158F} cells had similar PARP1 expression and PARylation signals (FIG. 11). Without wishing to be bound by any theory, these findings suggest that PARylation activity of PARP1 is not compromised, and that PARPi resistance mediated by p-Y158 PARP1 does not positively correlate with PARP1 enzymatic activity. Therefore, instead of altering PARylation activity of PARP1, FGFR3 appears to mediate PARPi resistance by phosphorylating PARP1 at Y158 residue to decrease PARP trapping caused by PARPi.

### Example 4

### Combinations of FGFRi and PARPi Inhibit Tumor Growth in Orthotopic Xenograft Models

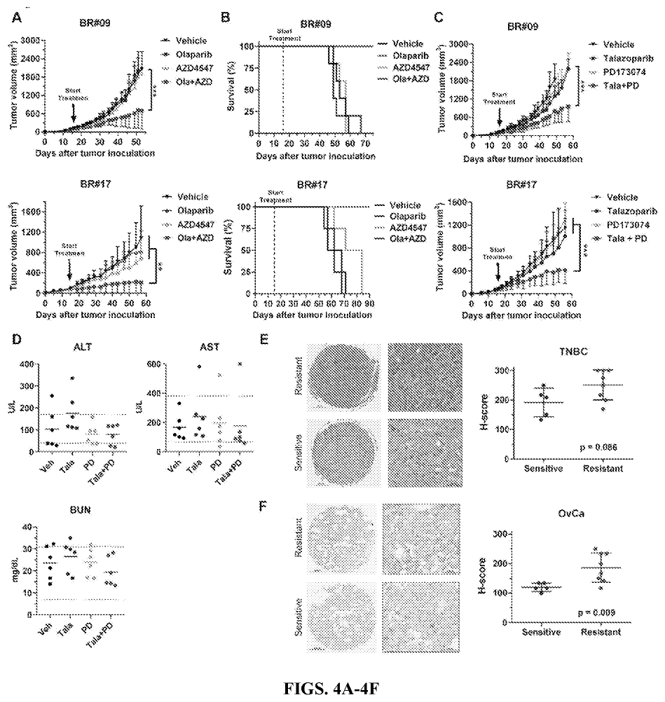

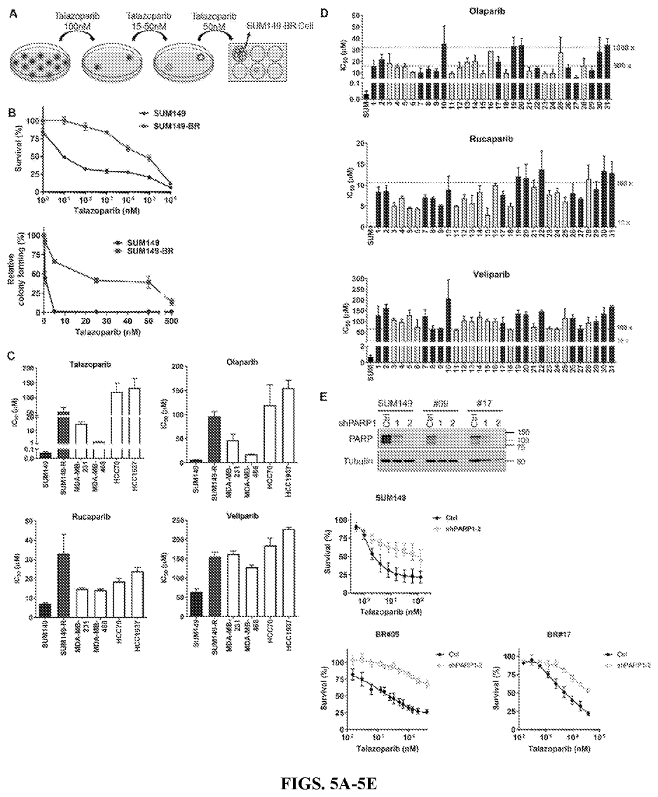

Xenograft mouse models were employed to evaluate the potency of the FGFRi and PARPi combination in suspending tumor growth in vivo. Inhibitor concentrations similar to or lower than the equivalent recommended dose for humans were used for treatments in mice (Litton, et al., 2018; Robson et al., 2017; de Bono, 2017; Paik et al., 2017). As shown in two BR xenograft models, olaparib alone did not inhibit tumor growth, and AZD4547 alone inhibited tumor growth in the BR# 17 model (p =0.0192 at day 57) but not in the BR# 09 model (FIG. 4A). However, the combination of olaparib and AZD4547 significantly inhibited tumor growth in both models (BR# 09, p <0.0001; BR# 17, p =0.0046 compared with AZD4547 alone and p <0.0001 compared with vehicle and olaparib alone; FIG. 4A). Therefore, the combination of olaparib and AZD4547 prolonged animal survival in both models (FIG. 4B).

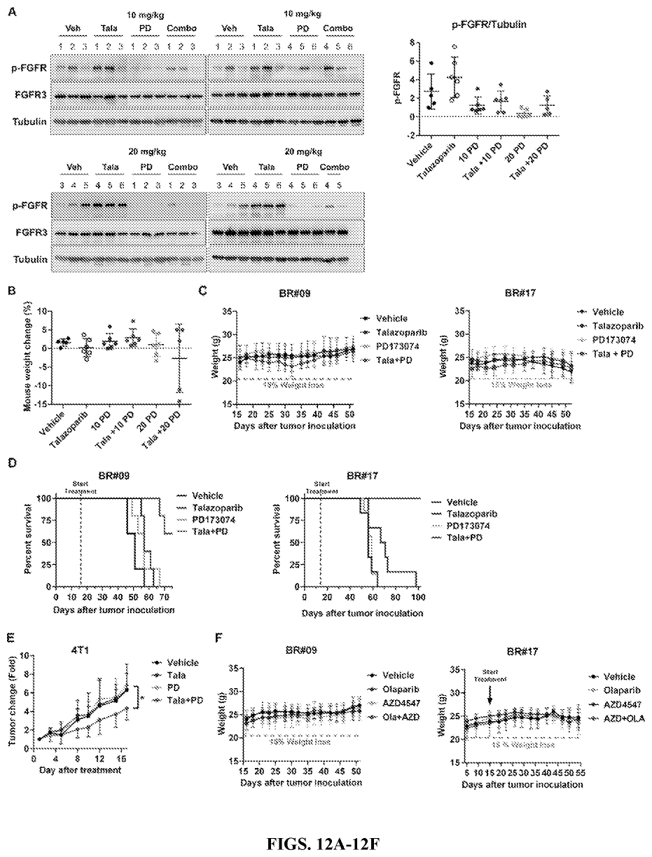

Because PD173074 has not been investigated in a clinical trial, the inventors titrated its concentration for animal use. With tumors harvested after 3 days of treatment, talazoparib induced FGFR3 phosphorylation and that talazoparib-induced FGFR phosphorylation was inhibited by PD173074 at a dose of 10 mg/kg per day and further inhibited by PD173074 at a dose of 20 mg/kg per day to less than the basal levels (FIG. 12A). However, one-third of mice treated with 20 mg/kg PD173074 per day combined with talazoparib experienced more than 10% weight loss (FIG. 12B). Therefore, a dose of 15 mg/kg PD173074 per day, which resulted in no loss of body weight (FIG. 12C), was used for the further animal studies. As expected, single-agent talazoparib or PD173074 did not inhibit tumor growth in either the BR# 09 or the BR# 17 models, but the combination of talazoparib and PD173074 significantly inhibited tumor growth (p <0.0001 in both models; FIG. 4C) and prolonged animal survival without inducing animal weight loss with long-term treatment (FIG. 12C-D).

### Example 5

### Combination of FGFRi and PARPi is Potent in Patient-Derived Xenograft (PDX) Models

The toxicity of the combination in a syngeneic 4T1 model was further evaluated, and the combination of talazoparib and PD173074 inhibited tumor growth more than the single-agent treatments did after 2 weeks of treatment, before mice were euthanized for toxicity tests (FIG. 12E). Blood chemical tests showed that the blood urea nitrogen, alanine aminotransferase, and aspartate aminotransferase levels in these animals were within the range of normal Balb/c mice (FIG. 4D), indicating that the kidney and liver functions of these mice were not damaged by the treatments. Meanwhile, animal weight loss was not observed in either model within 50 days of treatment (FIG. 12C and F), suggesting that the toxicity is tolerable in long-term treatment.

Because only a few breast cancer patients have received long-term treatment with PARPi, and because the PARPi resistance mechanisms reported are similar to those of platinum resistance (Noordermeer and van Attikum, 2019; Choi et al., 2016), TNBC (Choi et al., 2017) nd ovarian cancer PDX models with known PARPi or platinum resistance were used to study the clinical relevance of the combination of FGFRi and PARPi. Although FGFR signaling is already elevated in TNBC cells (Smith et al., 2017; Sharpe et al., 2011), in 13 TNBC PDX tissues, immunohistochemistry staining showed that talazoparib-resistant PDX models had higher H-scores for phosphorylated FGFR (H-score 251.4 ±51.1) than did talazoparib-sensitive models (phosphorylated FGFR H-score 191.7 ±48.6), although the difference was not statistically significant (p =0.086; FIG. 4E). Moreover, significantly higher H-scores for phosphorylated FGFR in 13 platinum-resistant ovarian cancer PDX models (H-score 186.5 ±49.6) were observed as compared with platinum-sensitive models (H-score 120.0 ±13.9, p =0.0093; FIG. 4F). These correlations support the idea that phosphorylated FGFR can serve as a biomarker to identify cancer patients with PARPi resistance and platinum resistance. Because p-Y158 PARP1 enhances PARP trapping, the potential of using p-Y158 PARP1 as a biomarker for PARPi resistance is worth evaluating when clinical samples become available.

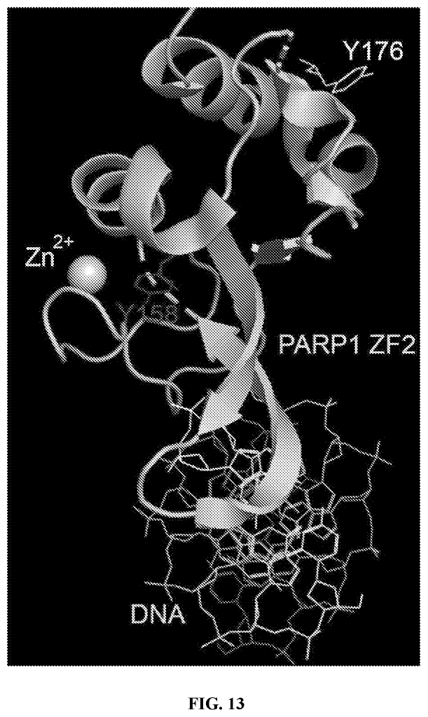

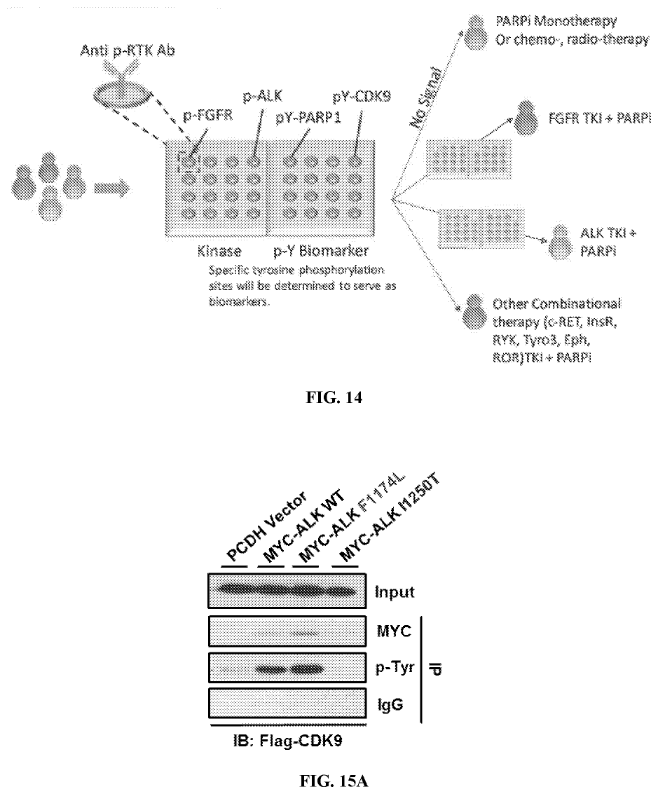

Although FGFR3 is not the only RTK that contributes to PARPi resistance (Nowsheen et c1.,.2012; Balaji et al., 2017; Han et al., 2019; Dong et al., 2019; Chu et al., 2020; Du, et al., 2016), it is the first one known to contribute to releasing PARP trapping. Structurally, the Y158 amino acid is adjacent to the PARP1 zinc finger 2 domain's zinc ion binding residues (C125, C128, H159, and C162) and the DNA interacting residues (L151/1156) (Langelier et al., 2011; Eustermann et al., 2011), indicating that Y158 may also be involved in protein structure stabilization (FIG. 13). However, detailed studies regarding the impact of p-Y158 on PARP1 conformation and dimerization should be further pursued.

MET and EGFR can contribute to PARPi resistance by phosphorylating the PARP1 catalytic domain at the Y907 residue (Han et al., 2019; Dong et al., 2019; Chu et al., 2020; Du, et al., 2016). p-Y907-PARP1 has higher enzymatic activity and a lower affinity to PARPi than un-phosphorylated PARP1, and thus, the combination of MET inhibitors and PARPi increases PARPi-induced DNA damage (Du, et al., 2016). In the current study, it was observed that the PARP1^{Y158F} mutant has similar enzymatic activity to PARP1^{WT}, and that the combination of FGFRi and PARPi prolongs PARP trapping without increasing the amount of DNA damage. Given that catalytic inhibition and PARP trapping are the two main aspects to consider for choosing an appropriate PARPi for drug combinations (Murai, et al., 2014), and that the PARP1 domains involved in the interaction with inhibitors vary among PARPi (Shen et al., 2015), the therapeutic efficacies resulting from combining inhibitors of these RTKs with PARPi may depend on the PARPi chosen for treatment.

Given the results of the current study, FGFR inhibition can reinstate PARPi sensitivity by inhibiting PARP1 phosphorylation at the Y158 residue, thus prolonging PARP trapping. The data show that phosphorylated FGFR can serve as a biomarker to indicate PARPi resistance. Although FGFRi and PARPi synergistically inhibited the growth of tumor cells in animal models, the toxicity of the combination was manageable. These data support the use of p-Y158-PARP1 and p-Y907-PARP1 antibodies in biomarker and/or RTK screening methods, and targeted PARPi combination strategies using these biomarker-kinase pairs may be utilized, e.g., in a personalized PARPi therapy to treat a cancer.

### Example 6

### ALK Interacts and Tyrosine Phosphorylates CDK9 at Y19

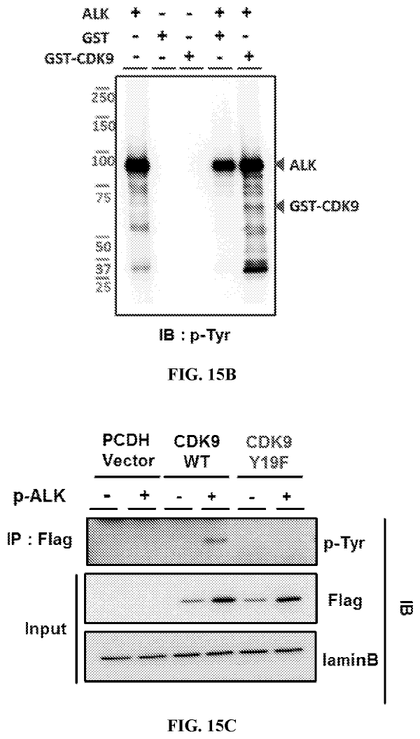

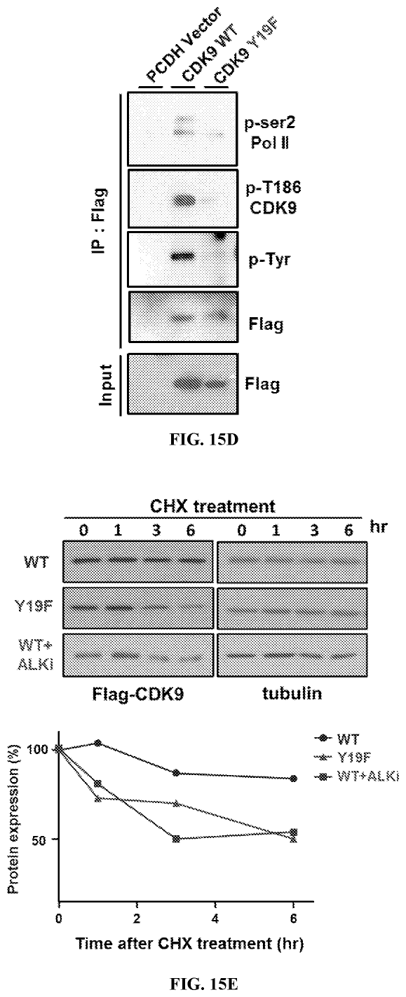

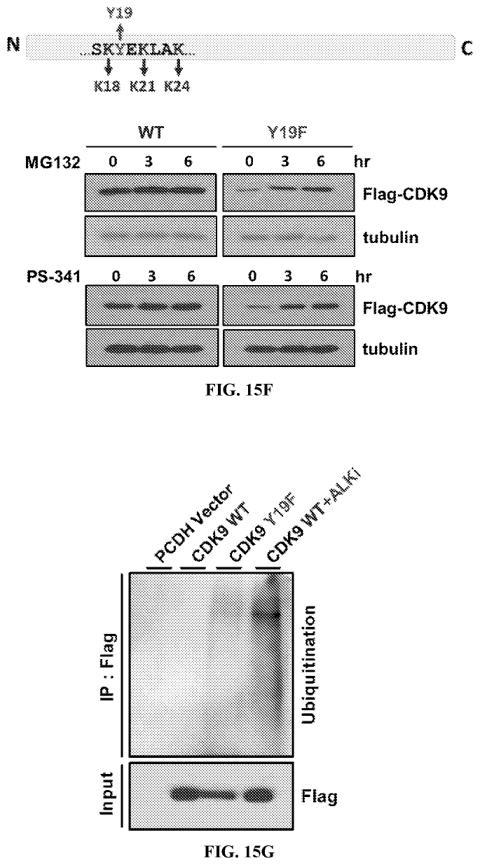

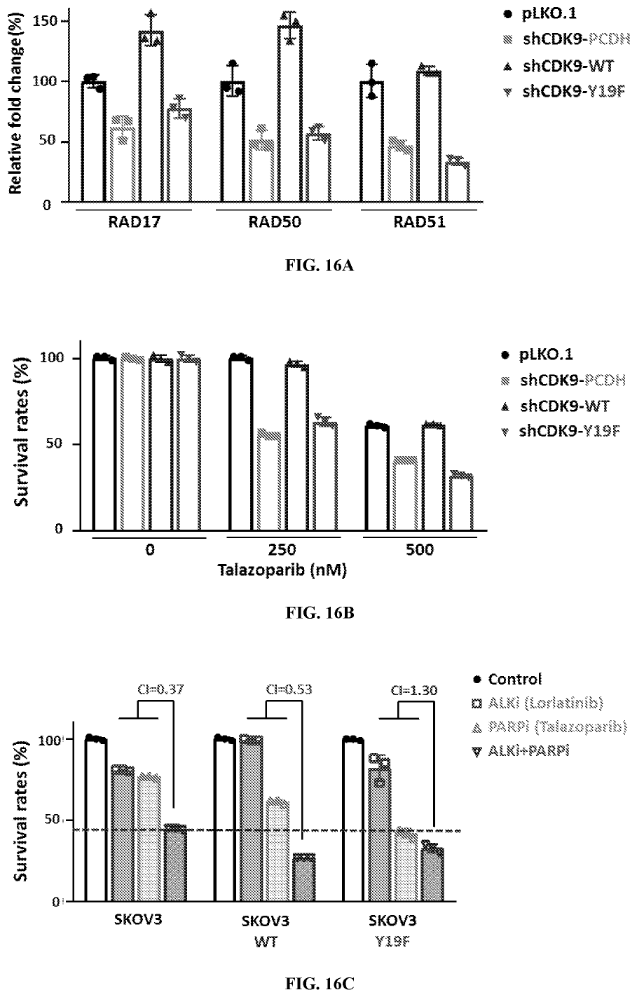

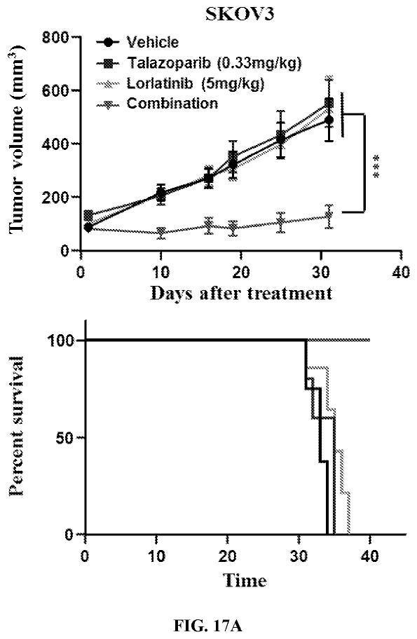

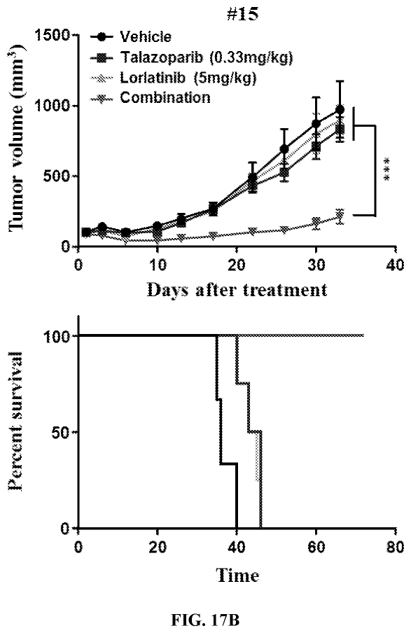

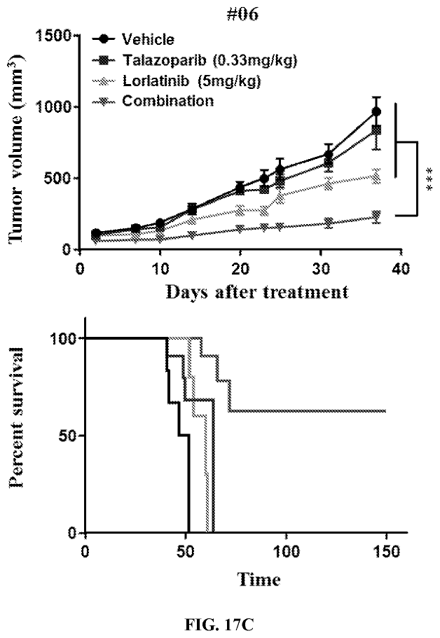

We discovered that ALK interacts and tyrosine phosphorylates CDK9 at Y19 (FIGS. 15A-C) and further demonstrated that p-Y19 is critical for kinase activity and protein stability of CDK9 (FIGS. 15D-G). Our results also showed that Y19F CDK9 fails to phosphorylate RNA Pol II at Ser2 and results in as HR-deficient phenotype via down-regulation of genes involved in HR (FIG. 15D and FIG. 16A). Indeed, synergistic inhibition of cellular proliferation was observed with combined blockade of PARP and ALK in cells with WT but not Y19F CDK9 (FIGS. 16B-C). Marked synergistic activity with dual PARP and ALK inhibition was also observed in PARPi-resistant tumor models in vivo (FIGS. 17A-C). Collectively, our data show that ALK contributes to PARPi resistance by phosphorylating CDK9 at Y19, thereby increasing the kinase activity and protein stability of CDK9 which in turn promotes HR-proficiency.

### Example 7

### Materials and Methods

### Cell Culture and Talazoparib-Resistant BR Cell Development

SUM149 cells (BioIVT) were maintained in F-12K medium (American Type Culture Collection [ATCC], 30-2004) supplied with 5% fetal bovine serum (FBS), 10 mM HEPES, 1 μg/ml hydrocortisone, 5 μg/ml insulin, and 100 units/ml penicillin with 100 μg/ml streptomycin (P/S). Other cell lines used were purchased from ATCC. MDA-MB-231, BT-549, MDA-MB-468, and MCF-7 cells were maintained in Dulbecco modified Eagle medium/F-12 medium (Caisson Laboratories) supplemented with 10% FBS and P/S. HCC70 and HCC1937 cells were maintained in RPMI 1640 medium (Corning) supplemented with 10% FBS and P/S. Cell lines were validated by short tandem repeat DNA fingerprinting using the AmpF_STR Identifiler kit according to the manufacturer's instructions (Applied Biosystems), and the profiles were matched to known ATCC fingerprints (ATCC.org) and to the Cell Line Integrated Molecular Authentication database (CLIMA) version 0.1.200808 (http://bioinformatics.istge.it/clima/). SUM149-BR cells were selected by treating SUM149 cells with 100 nM talazoparib for 5 consecutive days and then with 15-50 nM talazoparib until resistant cells grew into clones. Single clones were cultured in 50 nM talazoparib-containing complete F-12K medium until stably proliferating. The cells were then maintained without talazoparib.

### Reagents and Antibodies

Talazoparib (BMN-673), veliparib (ABT-888), PD173074, erdafitinib, and AZD4547 were purchased from Selleck Chemistry. Olaparib and rucaparib were purchased from LC Laboratories. AZD4547 and PD173074 for animal experiments were purchased from MedChemExpress. All inhibitors were dissolved in dimethyl sulfoxide (DMSO) or dimethylacetamide to make stock solution. Unless otherwise indicated, 100 nM talazoparib and 10 μM PD173074 were used for treatment of cells. Methyl methanesulfonate (MMS) was purchased from Sigma Aldrich and a final concentration of 0.01% MMS was used for treatment of cells.

The primary antibodies and dilution ratios for Western blot analysis used in the current study were as follows: rabbit anti-FGFR3 (# ab137084; 1:2,000) and rabbit anti-Histone H4 (# ab10158; 1:1,000) from Abcam; rabbit anti-PARP (# 9532S; 1:1,000) from Cell Signaling Technology; rabbit anti-actin (# A2066; 1:5,000), mouse anti-tubulin (# T5158; 1:5,000), mouse anti—phospho-histone H2A.X (Ser139; # 05-636; 1:1,000), mouse anti-HA (clone 12CA5; 1:1,000), and rabbit anti—phospho-FGFR (Tyr653/Tyr654; # 06-1433; 1:1000) from MilliporeSigma; and rabbit anti-lamin B1 (# sc-374015; 1:2,000) and mouse anti-GAPDH (# sc-32233; 1:1,000) from Santa Cruz Biotechnology.

### MTT Assay

Cells were seeded at a concentration of 1,000 cells/well in a 96-well plate and cultured overnight before treatments with inhibitors. Inhibitor-containing media was refreshed every 3 days for a 6-day treatment schedule. Cells were incubated with 0.5 mg/ml thiazolyl blue tetrazolium bromide (Sigma-Aldrich) for 2 hours, and formazan crystals were dissolved using DMSO. Optical density at 565 nm was measured and survival percentages were calculated by normalizing the optical density value of each treatment group to that of the control group, treated with DMSO only. Half-maximal inhibitory concentrations of inhibitors were calculated using the standard curve interpolate function in GraphPad Prism 8.

### Colony Formation Assay

SUM149 cells (600 cells/well), BR# 09 cells (800 cells/well), and BR# 17 cells (1,000 cells/well) were seeded into a 12-well plate 18 hours before treatments. Inhibitor-containing media was refreshed every 2 days. Cells were fixed using 4% paraformaldehyde after 10-14 days of treatment. Colonies were stained with 0.5% crystal violet before plates were imaged, and colony number was quantified using the Celigo imaging cytometer (Nexcelom Bioscience). Cell survival rate was calculated by normalizing the number of colonies in each well to that of the vehicle-treated well on the same culture plate.

### Immunoprecipitation and Western Blot Analysis

For immunoprecipitation, cells were treated and lysed with lysis buffer (20 mM Tris [pH 8.0], 137 mM NaCl, 1% Nonidet P-40, and 2 mM EDTA) before 500 μg of proteins was diluted to 500 !IL with lysis buffer and incubated with 2.5 μg primary antibodies overnight.

The precipitated protein complex was then washed and subjected to Western blot analysis performed as described previously (Chen, et al., 2019). Signals were detected using the ImageQuant 4000 system (GE Healthcare) and quantified using Image Studio Lite.

### Receptor Tyrosine Kinase Antibody Array

A Proteome Profiler Human Phospho-RTK Array Kit (R&D Systems, # ARY001B) was used according to the manufacturer's instructions. In brief, cells were treated with DMSO or 100 nM talazoparib overnight and then harvested for antibody array analysis. Signal data from the array were captured and analyzed as Western blot images. Signals on each array were normalized to the mean signal value of reference controls.

### Proximity Ligation Assay and Immunofluorescence Staining

Cells were treated for 1 hour with 0.01% MMS, 0.1 μM talazoparib, or 10 μM PD173074 as indicated before being fixed with 4% paraformaldehyde. PD173074 was introduced 2-4 hours before it was combined with other chemicals, to ensure that FGFR3 was inhibited while inducing DNA damage. The proximity ligation assay (Duolink In Situ Red, Sigma Aldrich) was performed following the manufacturer's instructions. Mouse anti-PARP1 (Sino Biological, # 11040-MM04) and rabbit anti-FGFR3 (Abcam, # ab137084) primary antibodies for the proximity ligation assay were diluted at a ratio of 1:500 and incubated with samples overnight at 4° C.

Immunofluorescence staining and confocal microscopy were performed as previously described (Chen, et al., 2019). Cells were treated with DMSO (solvent control), talazoparib (125 nM for BR# 09; 250 nM for BR# 17), PD173074 (10 μM), or the combination of talazoparib and PD173074 for the time indicated. Primary antibodies were diluted in 5% bovine serum albumin at a ratio of 1:500 for both anti—phospho-histone H2A.X and anti-FGFR3 and were incubated overnight. Secondary antibodies anti-mouse fluorescein isothiocyanate and anti-rabbit Texas Red were diluted at a 1:1,000 ratio in 5% bovine serum albumin. In both immunostaining and the proximity ligation assay, images of the cells were captured and analyzed with an LSM 710 laser confocal microscope and Zeiss Zen software (Carl Zeiss) and foci counting was performed using BlobFinder (Allalou and Wahlby, 2020).

### Cloning and Mutagenesis

FGFR3-expressing plasmid pDONR223 FGFR3 (Addgene plasmid # 23933) was a gift of Dr. William Hahn and Dr. David Root (Johannessen et al., 2010). FGFR3 was subcloned from pDONR223-FGFR3 into pCDH-CMV-MCS-EF1-Neo (System Biosciences) by amplifying the FGFR3 open reading frame with polymerase chain reaction. 3×Flag-tag was inserted by oligomer annealing. HA-tagged PARP1 expression plasmid was described previously (Du, et al., 2016). PARP1^{Y158F}- and PARP1^{Y176F}-expressing plasmids were generated using site-directed mutagenesis polymerase chain reaction and HA-PARP1 plasmid (Du, et al., 2016).

### RNA Interference and Stable Cell Lines

PARP1-targeting short hairpin RNAs (shRNAs; shPARP1-1: TRCN0000007928; shPARP1-2: TRCN0000356550) and FGFR3-targeting shRNAs (TRCN0000000371 and TRCN0000196809) were purchased from Sigma Aldrich. FGFR3-targeting shRNAs were subcloned into EZ-Tet-pLKO-Puro (Addgene plasmid # 85966), a gift from Dr. Cindy Miranti (Frank et al., 2017). Lentivirus particles were generated by transfecting HEK293T cells with pCMV-VSV-G (Addgene plasmid # 8454), pCMV-dR8.91, and shRNA plasmids, PARP1-expressing plasmids, or FGFR3-expressing plasmids in a 1:3:6 ratio. Scramble shRNA control plasmid pLKO.1 (Addgene plasmid # 1864) was a gift from David Sabatini and pCMV-VSV-G was a gift from Dr. Bob Weinberg. Stable cells were selected and maintained in the selection medium containing 1μg/ml puromycin (InvivoGen) or 500 μg/ml G418 (Thermo Fisher).

### Comet Assay

Cells were seeded into a 60-mm cell culture dish at least 18 hours before reaching 60% confluence for treatments. Cells were treated with 0.01% MMS, 100 nM talazoparib, and 10μ.M PD173074 as indicated for 1 hour before MMS removal. For cell release from MMS, culture medium was removed and cells were washed with ice-cold phosphate-buffered saline twice before freshly prepared inhibitor-containing medium was added for DNA repair. The alkaline comet assay was performed as described previously (Chen et al., 2011). DNA damage was further digested with 2U formamidopyrimidine [fapy]-DNA glycosylase (New England BioLabs, # M0240S) for 1 hour before electrophoresis (22 V, 300 mA, 20 minutes). Comet olive moment was measured using CometScore v1.5 (TriTek).

### PARP Trapping Assay

Chromatin-bound PARP1 was isolated as previously described (Okada, et al., 2006; Murai, et al., 2012) with some modifications. Cells were treated with or without 10 μM PD173074 for at least 4 hours before treatment with 100 nM talazoparib and 0.1% MMS. After treatment with MMS, cells were washed with ice-cold phosphate-buffered saline twice before incubation with fresh normal culture medium or PD173074-containing medium for the time indicated. Cells were then trypsin-harvested and lysed with HDG150 buffer before the chromatin fraction was incubated in HDG150 buffer with 5 mM CaCl₂ and 100 U/ml micrococcal nuclease for 1 hour at 4° C. Supernatant of chromatin fraction was then subjected to Western blot analysis.

### Mouse Models

Animal studies were performed following a protocol approved by The University of Texas MD Anderson Cancer Center Institutional Animal Care and Use Committee. Female nude mice were purchased from the Department of Experimental Radiation Oncology at MD Anderson. For BR# 09 and BR# 17 xenograft mouse models, two million cells were mixed with 50% (v/v) growth factor reduced Matrigel matrix (Corning) and inoculated into the mammary fat pads of 6- to 8-week-old female nude mice. For the 4T1 model, female Balb/c mice were purchased from Jackson Laboratory. A total of 50,000 4T1 cells were mixed with Matrigel matrix and inoculated into the mammary fat pad of 6-week-old female Balb/c mice. Inhibitors were dissolved in vehicle solvent containing 10% dimethylacetamide (Sigma-Aldrich), 5% Kolliphor HS 15 (Sigma-Aldrich), and 85% phosphate-buffered saline (Evans et al., 2017). Final concentrations of the inhibitors used in the mouse models are as follows: talazoparib (0.25 mg/kg per day), PD173074 (15 mg/kg per day), olaparib (40 mg/kg per day), and AZD4547 (8 mg/kg per day). Treatment with inhibitors started when tumor volumes reached a mean of 120 mm³. Mice were treated using oral gavage daily for 20 days followed by 3 days with no drugs to prevent severe weight loss. After the first cycle, treatment was continued on a schedule of 6 days on and 1 day off. Mouse weight and tumor volume were measured three times every week. Tumor volume was estimated using the following formula: volume (mm³) =length (mm)×width (mm)×0.5 width (mm), where length is the longest axis of the tumor. Mice were euthanized using CO₂ when the tumor volume reached 2,000 mm³. For the blood chemical test, mouse cardiac blood was collected by veterinarians in the Department of Veterinary Medicine and Surgery at MD Anderson after 16 days of treatment. Concentrations of alanine aminotransferase, aspartate aminotransferase, and blood urea nitrogen in Balb/c mice were referred to North American colonies of Charles River Balb/c mice.

Tyrosine 158 phosphorylated PARP1 Antibody Generation

Mouse anti-phospho-PARP1 Y158 antisera were generated by immunizing 20 mice with phospho-PARP1-Y158 KLH hot peptide (KLH-C-EKPQLGMIDRW-pY-HPG-S-FVKNREE; SEQ ID NO: 1) once every 2 weeks. Binding affinity specificities of the antisera were evaluated by enzyme-linked immunosorbent assay and Western dot blot with hot peptide (C-EKPQLGMIDRW-pY-HPG-S-FVKNREE; SEQ ID NO: 2) and cold peptide (C-EKPQLGMIDRW-Y-HPG-S-FVKNREE; SEQ ID NO: 3).

### Statistics

For Western blot signal quantification, signal intensities were analyzed using Image Studio Lite (version 5.2; LI-COR Biosciences). Signals of PARP1, PARylation, FGFR3, and phosphorylated FGFR were first normalized to house-keeping proteins (tubulin, actin, or GAPDH) of each sample before being normalized to the control groups. Every independent experiment repeat was quantified individually. Fold changes in Western blot signals were analyzed by a nonparametric Friedman test using the GraphPad Prism 8.0 software. A p value of less than 0.05 was considered statistically significant: *p <0.05; **p <0.002; ***p <0.001. Combination index experiments were designed according to the Chou-Talalay method³⁹, and results were calculated using Compusyn software.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 7,928,105
U.S. Pat. No. 8,124,606
U.S. Pat. No. 8,236,802
U.S. Pat. No. 8,450,323
WO 2006/110816
WO 2008/083027
WO 2011/014681
WO 2007/084532
Allalou and Wahlby, Comput Methods Programs Biomed 94, 58-65, 2009.
Anders et al., Clinical Cancer Research, 16:4702-4710, 2010.
Bagai et al., IDrugs Investig. Drugs 1; 13:404-414, 2010.
Balaji et al., Mol Cancer Res 15, 45-58, 2017.
Bhullar et al., Mol Cancer 17, 48, 2018.
Bryant et al., 2005.
Carey et al., Nature reviews. Clinical Oncology, 7:683-692, 2010.
Cesaire et al., Int J Mol Sci 19, 2018.
Chen et al., Toxicol Sci 122, 339-348, 2011.
Chen, et al., J Blot Chem 294, 8516-8528, 2019.
Choi et al., Cell Rep 14, 429-439, 2016.
Chou and Talalay Adv Enzyme Regul 22: 27-55, 1984.
Chou, Cancer Res 70, 440-446, 2010.
Chou, Pharmacol Rev 58, 621-681, 2006.
Chu et al., Am J Cancer Res 10, 648-661, 2020.
Comoglio et al., Nat. Rev. Drug Discov.; 7:504-516, 2008.
Davies and Tournier, Biochem. Soc. Trans.; 40:85-89, 2012.
Davies et al., Nature.; 417:949-954, 2002.
de Bono et al., Cancer Discov 7, 620-629, 2017.
Demetri et al., Lancet; 368:1329-1338, 2006.
Donawho et al., Clin. Cancer Res., 13:2728-2737, 2007.
Dong et al., Cancer Res 79, 819-829, 2019.
Drean, et al., Crit Rev Oncol Hematol 108, 73-85, 2016.
Du et al., Nat Med 22: 194-201, 2016.
Eustermann et al., J Mol Biol 407, 149-170, 2011.
Evans et al., Clin Cancer Res 23, 6468-6477, 2017.
Fabbro and Manley, Curr. Opin. Investig. Drugs (Lond. Engl.); 2:1142-1148, 2001.
Fang et al., Cancer Cell 35, 851-867 e857, 2019.
Farmer et al., Nature, 434:917-921, 2005.
Frank et al., BMC Biotechnol 17, 24, 2017.
Gelmon et al., Lancet Oncol., 12:852-861, 2011.
Gibson and Kraus, Nat. Rev. Mol. Cell Biol., 13:411-424, 2012.
Hahn et al., J. Vet. Intern. Med.; 22:1301-1309, 2008.
Hampson et al., J. Immunol. Methods, 360:30-38, 2010.
Han et al., Am J Cancer Res 9, 608-618, 2019.
Hilberg et al., Cancer Res.; 68:4774-4782, 2008.
Hubbard and Miller, Curr. Opin. Cell Blot; 19:117-123, 2007.
Huynh et al., Clin. Cancer Res., 14:6146-6153, 2008.
Irani et al., Science, 275:1649-1652, 1997.
Johannessen et al., Nature 468, 968-972, 2010.
Knights and Cook, Pharmacol. Ther. 125:105-117, 2010.
Koch and Claesson-Welsh, Cold Spring Harbor Per spect. Med., 2012.
Kumar et al., Mol. Cancer Ther.; 6:2012-2021, 2007.
Kummar et al., J Clin. Oncol., 27:2705-2711, 2009.
Langelier et al., J Biol Chem 286, 10690-10701, 2011.
Lemmon and Schlessinger, Cell; 141:1117-1134, 2010.
Li and Hristova, Biochemistry;45:6241-6251, 2006.
Liedtke et al., Journal of Clinical Oncology, 26:1275-1281, 2008.
Lindahl, Nature, 362:709-715, 1993.
Linklater et al., Oncotarget 7: 69903-69915, 2016.
Litton et al., N Engl J Med 379, 753-763, 2018.
Lord and Ashworth, Nat. Med., 19:1381-1388, 2013.
Luo and Kraus, Genes Dev., 26:417-432, 2012.
Marek et al., Mol. Pharmacol.; 75:196-207, 2009.
McDermott et al., Bioorg. Med. Chem.; 13:4835-4841, 2005.
Montemurro et al., Oncologist; 11:318-324, 2006.
Motzer et al., J. Clin. Oncol.; 27:3584-3590, 2009.
Murai and Pommier, Annual Review of Cancer Biology 3, 131-150, 2019.
Murai, et al., Cancer Res 72, 5588-5599, 2012.
Murai et al., J Pharmacol Exp Ther 349, 408-416, 2014.
Noordermeer and van Attikum, Trends Cell Biol 29, 820-834, 2019.
Norquist et al., J Clin Oncol 29, 3008-3015, 2011.
Nowsheen et al., PLoS One 7, e46614, 2012.
Okada et al., Nat Cell Biol 8, 446-457, 2006.
O'Shaughnessy et al., Journal of Clinical Oncology, 29:abstr 1007, 2011a.
O'Shaughnessy et al., The New England Journal of Medicine, 364:205-214, 2011b.
Paik et al., Clin Cancer Res 23, 5366-5373, 2017.
Pilie et al., A. Nat Rev Clin Oncol 16, 81-104, 2019.
Pommier et al., Sci Transl Med 8, 362ps317, 2016.
Previdi et al., Mol. Cancer Ther.; 11:214-223, 2012.
Radisky et al., Nature, 436:123-127, 2005.
Robson et al., N Engl J Med 377, 523-533, 2017.
Romano et al., Nucleic Acids Res 37: D925-932, 2009.
Rouleau et al., Nature Reviews Cancer, 10:293-301, 2010.
Sarker et al., Clin. Cancer Res.; 14:2075-2081, 2008.
Schiering et al., Proc. Natl. Acad. Sci. USA.; 100:12654-12659, 2003.
Sharma and Settleman, Genes Dev 21, 3214-3231, 2007.
Sharpe et al., Clin Cancer Res 17, 5275-5286, 2011.
Shen et al., J Pharmacol Exp Ther 353, 446-457, 2015.
Smith et al., Breast Cancer Res 19, 65, 2017.
Trachootham et al., Nat. Rev. Drug Discov., 8:579-591, 2009.
Tripathy et al., J. Clin. Oncol; 22:1063-1070, 2004.
Trudel et al., Blood.; 105:2941-2948, 2008.
Turashvili et al., Mod. Pathol., 24:64-81, 2011.
Tutt et al., Lancet, 376:235-244, 2010.
Underiner et al., Anti-Cancer Agents Med. Chem.; 10:7-27, 2010.
Waks et al., Ann Oncol, 2020.
Wander et al., J. Clin. Investig.; 121:1231-1241, 2011.
Yap et al., Am Soc Clin Oncol Educ Book 39, 185-195, 2019.

What is claimed is:

1. A method of treating a cancer in a mammalian subject comprising administering to the subject a therapeutically effective amount of a PARP1 inhibitor and a receptor tyrosine kinase (RTK) inhibitor, wherein the RTK inhibitor is not a MET inhibitor.

2. The method of claim 1, wherein the RTK inhibitor is a fibroblast growth factor receptor (FGFR) inhibitor, insulin receptor (InsR) inhibitor, Tyro3 inhibitor, anaplastic lymphoma kinase (ALK) inhibitor, Ret proto-oncogene (c-RET) inhibitor, ephrin receptor (Eph) inhibitor, RYK inhibitor, or receptor tyrosine kinase like orphan receptor (ROR) inhibitor.

3. The method of claim 1, wherein the RTK inhibitor is a FGFR inhibitor or an ALK inhibitor.

4. The method of claim 1, wherein the patient is determined to have a cancer expressing Tyr158 and/or Try176 phosphorylated PARP1, and wherein the method comprises administering to the patient a therapeutically effective amount of a combination of a PARP1 inhibitor and an FGFR inhibitor.

5. The method of claim 1, wherein the patient is determined to have a cancer expressing phosphorylated CDK9, and wherein the method comprises administering to the patient a therapeutically effective amount of a combination of a PARP1 inhibitor and an ALK inhibitor.

6. The method of any one of claims 1-5, wherein the cancer is a breast cancer, renal cancer, lung cancer, ovarian cancer, colon cancer, prostate cancer or pancreatic cancer.

7. The method of claim 6, wherein the breast cancer is a triple-negative breast cancer.

8. The method of any one of claims 1-7, wherein the PARP1 inhibitor is olaparib, ABT-888 (Veliparib), BSI-201 (Iniparib), BMN 673, Rucaparib (AG-014699, PF-01367338), AG14361, INO-1001, A-966492, PJ34, MK-4827, or Fluzoparib.

9. The method of any one of claims 3-8, wherein the FGFR inhibitor is PD173074, AZD4547, Brivanib (BMS-540215), CHIR-258 (TKI-258) , or LY2874455, Dovitanib, or JNJ42756493 (erdafitinib).

10. The method of any one of claims 3-8, wherein the ALK inhibitor is crizotinib, ceritinib, alectinib, or lorlatinib.

11. The method of any one of claims 1-10, wherein the PARP1 inhibitor is administered concurrently with or essentially simultaneously with the RTK inhibitor.

12. The method of any one of claims 1-11, wherein the patient has previously undergone at least one round of anti-cancer therapy.

13. The method of any one of claims 1-12, wherein the subject is a human.

14. The method of any one of claims 1-13, further comprising administering a second anticancer therapy.

15. The method of claim 14, wherein the second anticancer therapy is a surgical therapy, chemotherapy, radiation therapy, cryotherapy, hormonal therapy, toxin therapy, immunotherapy, or cytokine therapy.

16. A method of predicting resistance of a cancer in a patient to a PARP1 inhibitor comprising assaying a cancer sample to detect or determine a phosphorylation status of PARP1 Tyr158, PARP1 Tyr176, and/or CDK9 in the cancer sample; wherein increased phosphorylation of PARP1 Tyr158, PARP1 Tyr176, and/or CDK9 in the cancer sample indicates that the cancer has an increased risk of resistance to a PARP1 inhibitor.

17. The method of claim 16, wherein PARP1 Tyr158 or Tyr176 is phosphorylated in the cancer sample.

18. The method of any one of claims 16-17, wherein CDK9 is phosphorylated in the cancer sample.

19. The method of any one of claims 16-18, wherein the method further comprises reporting whether the patient has a cancer that is resistant to a PARP1 inhibitor.

20. The method of claim 19, wherein the reporting comprises preparing a written or oral report.

21. The method of claim 19, further comprising reporting to the patient, a doctor, a hospital, or an insurance provider.

22. The method of any one of claims 16-21, wherein the assaying comprises measuring the level of phosphorylation of PARP1 Tyr158, PARP1 Tyr176, and CDK9.

23. The method of any one of claims 16-22, wherein the assaying comprises contacting the sample with an antibody that binds specifically to phosphorylated PARP1 Tyr158, PARP1 Tyr176, and/or CDK9.

24. The method of any one of claims 16-23, wherein the assaying comprises or consists of a Western blot, ELISA, immunoprecipitation, radioimmunoassay, or immunohistochemical assay.

25. The method of any one of claims 16-24, wherein the patient has a cancer that is resistant to a PARP1 inhibitor therapy, and wherein the method further comprises identifying the patient as a candidate for a combination of a PARP1 inhibitor and an RTK inhibitor.

26. The method of any one of claims 16-25, wherein the PARP1 inhibitor is olaparib, ABT-888 (Veliparib), BSI-201 (Iniparib), Talazoparib (BMN 673), Rucaparib (AG-014699, PF-01367338), AG14361, INO-1001, A-966492, PJ34, MK-4827, or Fluzoparib.

27. The method of claim 25, wherein if PARP1 Tyr158 or Tyr176 is phosphorylated in the cancer sample, then the RTK inhibitor is a FGFR inhibitor.

28. The method of claim 27, wherein the FGFR inhibitor is PD173074, AZD4547, Brivanib (BMS-540215), CHIR-258 (TKI-258), LY2874455, Dovitanib, or JNJ42756493 (erdafitinib).

29. The method of claim 25, wherein if CDK9 is phosphorylated, then the RTK inhibitor is an ALK inhibitor.

30. The method of claim 29, wherein the ALK inhibitor is crizotinib, ceritinib, alectinib, or lorlatinib.

31. A method of selecting a drug therapy for a cancer patient comprising:
(a) assaying cancer sample from the patient to determine a phosphorylation status of PARP1 Tyr158 and/or PARP1 Tyr176 in the sample; and
(b) selecting a combination of a PARP1 inhibitor and an FGFR inhibitor as the drug therapy if PARP1 Tyr158 and/or PARP1 Tyr176 is determined to be phosphorylated.

32. A method of selecting a drug therapy for a cancer patient comprising:
(a) assaying cancer sample from the patient to determine a phosphorylation status of CDK9 in the sample; and
(b) selecting a combination of a PARP1 inhibitor and an ALK inhibitor as the drug therapy if CDK9 is determined to be phosphorylated.

33. A method of sensitizing a cancer to a PARP1 inhibitor-based anticancer therapy comprising administering an effective amount of an RTK inhibitor to a patient having the cancer, wherein the RTK inhibitor is not a MET inhibitor.

34. The method of claim 33, further comprising administering a PARP1 inhibitor-based anticancer therapy to the subject.

35. The method of claim 34, wherein the PARP1 inhibitor-based anticancer therapy is administered concurrently with or essentially simultaneously with the RTK inhibitor.

36. The method of any one of claims 33-35, wherein the RTK inhibitor is a FGFR inhibitor, ALK inhibitor, TYRO3 inhibitor, InsR inhibitor, c-RET inhibitor, Eph inhibitor, RYK inhibitor, or ROR inhibitor.

37. The method of any one of claims 33-35, wherein the RTK inhibitor is a FGFR inhibitor or ALK inhibitor.

38. The method of claim 37, wherein the FGFR inhibitor is PD173074, AZD4547, Brivanib (BMS-540215), CHIR-258 (TKI-258), LY2874455, Dovitanib, or JNJ42756493 (erdafitinib).

39. The method of any one of claims 37-38, wherein the ALK inhibitor is crizotinib, ceritinib, alectinib, or lorlatinib.

40. The method of any one of claims 34-39, wherein the PARP1 inhibitor is olaparib, ABT-888, BSI-201, BMN 673, Rucaparib (AG-014699, PF-01367338), AG14361, INO-1001, A-966492, PJ34, MK-4827, or Fluzoparib.

41. A composition comprising a PARP1 inhibitor and an RTK inhibitor for use in treating a cancer in a patient, wherein the RTK inhibitor is not a MET inhibitor.

42. The composition of claim 41, wherein the PARP1 inhibitor is olaparib, ABT-888, BSI-201, BMN 673, Rucaparib (AG-014699, PF-01367338), AG14361, INO-1001, A-966492, PJ34, MK-4827, or Fluzoparib.

43. The composition of any one of claims 41-42, wherein the RTK inhibitor is a FGFR inhibitor, ALK inhibitor, Tyro3 inhibitor, InsR inhibitor, c-RET inhibitor, Eph inhibitor, RYK inhibitor, or ROR inhibitor.

44. The method of claim 43, wherein the RTK inhibitor is a FGFR inhibitor or ALK inhibitor.

45. The method of claim 44, wherein the FGFR inhibitor is PD173074, AZD4547, Brivanib (BMS-540215) or CHIR-258 (TKI-258) , or LY2874455, Dovitanib, or JNJ42756493 (erdafitinib).

46. The method of any one of claims 41-45, wherein the composition is formulated for parenteral, intravenous, intratumoral, subcutaneous, or oral administration.

47. A composition comprising an antibody that specifically binds to either: a Tyr158-phosphorylated PARP1 protein or a Tyr176-phosphorylated PARP1 protein.
